(19) United States
(12) Patent Application Publication
(10) Pub. No.: US 2018/0289858 A1
(43) Pub. Date: Oct. 11, 2018

(54) Bifunctional Bioactive Antibacterial Coatings, and Process for Coating Implant Surfaces Therewith

(71) Applicant: The University of Toledo, Toledo, OH (US)

(72) Inventors: Sarit B. Bhaduri, Toledo, OH (US); Vijay K. Goel, Toledo, OH (US); Yufu Ren, Toledo, OH (US); Prabaha Sikder, Toledo, OH (US)

(73) Assignee: The University of Toledo, Toledo, OH (US)

(21) Appl. No.: 15/946,851

(22) Filed: Apr. 6, 2018

Related U.S. Application Data
(60) Provisional application No. 62/482,906, filed on Apr. 7, 2017.

(51) Int. Cl. A61L 27/32; A61L 27/30; A61L 27/54; A61L 27/06

(52) U.S. Cl. CPC A61L 27/32; A61L 27/306; A61L 27/54; A61L 27/06; A61L 2420/06; A61L 2430/12; A61L 2300/104; A61L 2300/404; A61L 2420/02; A61L 2430/02

(57) ABSTRACT

Bifunctional coatings and implants coated with the same are described, as well as methods and compositions for making the same.

### RELATED APPLICATIONS

This application claims priority to U.S. Provisional Application Ser. No. 62/482,906 filed under 35 U.S.C. § 111(b) on Apr. 7, 2017, the disclosure of which is incorporated herein by reference in its entirety.

### STATEMENT REGARDING FEDERALLY SPONSORED RESEARCH

This invention was made with government support under Grant Number IIP1312211 awarded by the National Science Foundation. The government has certain rights in this invention.

### BACKGROUND OF THE INVENTION

There is a need in the art for improved capabilities of treating bacterial infections in orthopedics. An important example is Osteomyelitis (OM), an inflammation of the bone and surrounding areas due to bacterial infection. There is a global effort in the treatment of this condition, which affects both the young and old, in civilian as well as in military situations.

Furthermore, with the aging of the baby boomers in the U.S., more than 4.4 million people have received at least one internal fixation device and more than 1.3 million people have been fitted with an artificial joint. There are two important requirements for the successful performance of implants: strong osseointegration at the bone-implant interface, and prevention of recurrent bacterial infections. Infections are as high as 0.75% in hip implants and 1.5% in case of knee implants, both of which represent a significant number of cases. In many cases, bacterial infections can be combated by the host's immunological defense and systemic antibiotic delivery. In spite of that, surgical site infection (SSI) can eventually develop, making the scenario critical. Infections can result in reduced lifespan of the prosthesis, increased failure rate of the prosthesis, second surgeries, added medical costs, increased morbidity of tissues at the site, and subsequent patient dis-satisfaction. There are two major problems dealing with SSI. First, it may not be possible for the systemically delivered antibiotic to reach the depth of the implantation. Second, it is easy for the bacteria to create implant adhering biofilms due to their self-producing polysaccharides, and can therefore exhibit superior resistance to even larger doses of antibiotics. According to the National Institutes of Health, up to 80% of human bacterial infections involve biofilm-associated microorganisms.

Antibiotics have been routinely used to treat such infections. However, along with the development of newer drugs, bacteria became exposed to antibiotics and in turn developed a rapid resistance against them. The total cost of developing a new antibiotic drug is prohibitively high. Thus, an important alternate strategy is to modify implant surfaces with bactericidal elements. Metal ions of Ag, Zn, Cu, Mg have shown satisfactorily positive bactericidal effects in certain situations. Among them, Ag is proven to be a satisfactory bactericidal agent because of its capability to interfere and disrupt the breathing mechanisms of bacteria. Numerous situations have highlighted the application of Ag as an integral and efficient bactericidal element in coating implants. Table 1 presents a comparison of the important bactericidal agents.

TABLE 1
Comparison of some antibacterial agents

| Properties | ZnO | Cu | Ag (most preferred) |
|---|---|---|---|
| Functionality | Promising in | Strong | Very strong |
| | controlling | antimicrobial | antimicrobial |
| | spread and | properties | efficacy and antiviral |
| | colonization in | | properties. |
| | potential | | |
| | pathogens | | |
| Synthesis | Requires | Cost-effective | Reasonable |
| | control during | compared | |
| | synthesis. | to Ag. | |
| | Limited to | Gets complicated | |
| | small scale | due to | |
| | production | rapid oxidation. | |
| Cost | Cheapest of all. | Cheaper than Ag | Most expensive |
| Toxicity | More toxic | More toxic | Low toxicity to |
| | than Ag. | than Ag | human/mammalian |
| | | | cells. |
| Uses | Active | Treatment of | Mainly biomedical |
| | ingredient in | headaches, | applications, |
| | antibacterial | burns, intestinal | diagnosis, |
| | creams, | worms, | drug delivery, |
| | lotions and | ear infections, | implant |
| | ointments | and hygiene | coating. |

Antibacterial coatings and deposition techniques have been developed to obtain successful performance of implants. However, these deposition techniques have drawbacks, including a lack of cost-effectiveness for industrial viability, and the use of high temperatures with concomitant degradation of substrate surfaces especially in polymers (which are low melting point materials). Similarly, plasma deposition is a considerably complex process. Thus, there is a need for a simple, cost-effective industrially viable deposition technique suitable for all kinds of implants.

### SUMMARY OF THE INVENTION

Provided herein is a composition comprising a phosphate material doped with Ag, where the phosphate material is selected from the group consisting of hydroxyapatite and newberyite, and where the composition is characterized by an X-ray diffraction pattern with no peaks corresponding to elemental Ag. In certain embodiments, the composition is a coating on a substrate comprising Ti. In particular embodiments, the composition further includes a TiO₂ phase on the substrate. In particular embodiments, the TiO₂ phase is rutile.

Further provided herein is a method of making a coating on a substrate, the method comprising immersing a substrate in a coating solution comparing a source of silver ions, and exposing the immersed substrate to microwave radiation so as to form a silver-containing coating on the substrate, where the silver-containing coating comprises calcium phosphate or magnesium phosphate. Also provided are products of the method.

In certain embodiments, the source of silver ions comprises a water-soluble silver compound selected from the group consisting of: silver nitrate (AgNO₃), silver chloride (AgCl), silver fluoride (AgF), silver acetate (AgC₂H₃O₂), silver permanganate (AgMnO₄), silver sulfate (Ag₂SO₄), silver nitrite (AgNO₂), silver bromated (AgBrO₃), silver salicylate (HOC₆H₄COOAg), silver iodate (AgIO₃), silver dichromate (Ag₂Cr₂O₇), silver chromate (Ag₂CrO₄), silver carbonate (Ag₂CO₃), silver citrate (C₆H₈Ag₃O₇), silver phosphate (Ag₃PO₄), silver stearate (Ag[(CH₃)(CH₂)]₁₆)CO₂), silver (I) oxide (Ag₂O), silver sulfide (Ag₂S), silver bromide (AgBr), silver iodide (AgI), silver cyanide (AgCN), and combinations thereof. In certain embodiments, the coating solution further comprises a pH buffer.

In certain embodiments, the coating solution further comprises a source of calcium ions and a source of phosphate ions. In particular embodiments, the source of calcium ions comprises a water-soluble calcium-containing compound selected from the group consisting of calcium nitrate [Ca(NO₃)₂], calcium chloride (CaCl₂), calcium bromide (CaBr₂), calcium acetate [(CH₃COO)₂Ca], calcium citrate, calcium iodide (CaI₂), calcium lactate, calcium gluconate, calcium fumarate, calcium oxide (CaO), calcium hydroxide [Ca(OH)₂], calcium benzoate, calcium formate, calcium butyrate, calcium isobutyrate, calcium malate, calcium maleate, calcium propionate, calcium valerate, hydrates thereof, and combinations thereof. In particular embodiments, the source of phosphate ions is selected from the group consisting of: phosphoric acid (H₃PO₄); ammonium dihydrogen phosphate (NH₄H₂PO₄); Group I salts of dihydrogen phosphate, including sodium dihydrogen phosphate (NaH₂PO₄), lithium dihydrogen phosphate (LiH₂PO₄), potassium dihydrogen phosphate (KH₂PO₄), cesium dihydrogen phosphate (CsH₂PO₄), and rubidium dihydrogen phosphate (RbH₂PO₄); and combinations thereof.

In certain embodiments, the coating solution further comprises a source of magnesium ions and a source of phosphate ions. In particular embodiments, the source of magnesium ions comprises a water-soluble magnesium-containing compound selected from the group consisting of: magnesium nitrate [Mg(NO₃)₂], magnesium chloride (MgCl₂), magnesium sulfate (MgSO₄), magnesium acetate, magnesium benzoate, magnesium bromide (MgBr₂), magnesium chromate, magnesium bromate, magnesium citrate, magnesium formate, magnesium hexafluorosilicate, magnesium iodide (MgI₂), magnesium lactate, magnesium perchlorate, magnesium salicylate, magnesium sulfite, magnesium tartrate, magnesium thiosulfate, hydrates thereof, and combinations thereof. In particular embodiments, the source of phosphate ions is selected from the group consisting of: phosphoric acid (H₃PO₄), ammonium dihydrogen phosphate (NH₄H₂PO₄), sodium dihydrogen phosphate (NaH₂PO₄), lithium dihydrogen phosphate (LiH₂PO₄), potassium dihydrogen phosphate (KH₂PO₄), cesium dihydrogen phosphate (CsH₂PO₄), rubidium dihydrogen phosphate (RbH₂PO₄), and combinations thereof.

In certain embodiments, the substrate is an orthopedic or dental implant. In certain embodiments, the substrate comprises titanium, gold, silver, stainless steel, tantalum, platinum, tungsten, palladium, chromium, cobalt, alumina, zirconia, polyetheretherketone (PEEK), ultra high molecular weight polyethylene (UHMWPE), polymethyl methacrylate (PMMA), polylactic acid (PLA), polylactic acid co-polymer with glycolic acid (PLGA), polycaprolactone (PCL), polyhydroxyalkanoates (PHA) such as poly-3-hydroxybutyrate (P3HB), or mixtures, composites, or combinations thereof. In certain embodiments, the substrate comprises Ti6Al4V. In certain embodiments, the method further comprises drying the silver-containing coating. In certain embodiments, the method further comprises an etching step prior to the immersing, where the substrate is etched with NaOH. In particular embodiments, the method further comprises rinsing the etched substrate with water, drying the rinsed substrate, and cooling the dry substrate to room temperature prior to the immersing.

Further provided herein is an orthopedic or dental implant comprising a substrate, and a bifunctional coating on the substrate, where the bifunctional coating comprises a silver-doped calcium phosphate or a silver-doped magnesium phosphate, the bifunctional coating being characterized by an X-ray diffraction pattern having no peaks attributal to elemental silver, where the bifunctional coating is antibacterial and capable of osseointegration. In certain embodiments, the bifunctional coating is not cytotoxic. In certain embodiments, the silver-doped calcium phosphate is silver-doped hydroxyapatite, or the silver-doped magnesium phosphate is silver-doped newberyite. In certain embodiments, the substrate comprises titanium, gold, silver, stainless steel, tantalum, platinum, tungsten, palladium, chromium, cobalt, alumina, zirconia, polyetheretherketone (PEEK), ultra high molecular weight polyethylene (UHMWPE), polymethyl methacrylate (PMMA), polylactic acid (PLA), polylactic acid co-polymer with glycolic acid (PLGA), polycaprolactone (PCL), polyhydroxyalkanoates (PHA) such as poly-3-hydroxybutyrate (P3HB), or mixtures, composites, or combinations thereof.

Further provided herein is a solution for coating a substrate, the solution comprising a source of calcium ions, a source of phosphate ions, and a source of silver ions, where the ratio of calcium ions to phosphate ions in the solution is about 1.67. In certain embodiments, the source of calcium ions comprises a water-soluble calcium-containing compound selected from the group consisting of: calcium nitrate [Ca(NO₃)₂], calcium chloride (CaCl₂), calcium bromide (CaBr₂), calcium acetate [(CH₃COO)₂Ca], calcium citrate, calcium iodide (CaI₂), calcium lactate, calcium gluconate, calcium fumarate, calcium oxide (CaO), calcium hydroxide [Ca(OH)₂], calcium benzoate, calcium formate, calcium butyrate, calcium isobutyrate, calcium malate, calcium maleate, calcium propionate, calcium valerate, hydrates thereof, and combinations thereof. In certain embodiments, the source of silver ions comprises a water-soluble silver compound selected from the group consisting of: silver nitrate (AgNO₃), silver chloride (AgCl), silver fluoride (AgF), silver acetate (AgC₂H₃O₂), silver permanganate (AgMnO₄), silver sulfate (Ag₂SO₄), silver nitrite (AgNO₂), silver bromated (AgBrO₃), silver salicylate (HOC₆H₄COOAg), silver iodate (AgIO₃), silver dichromate (Ag₂Cr₂O₇), silver chromate (Ag₂CrO₄), silver carbonate (Ag₂CO₃), silver citrate (C₆H₈Ag₃O₇), silver phosphate (Ag₃PO₄), silver stearate (Ag[(CH₃)(CH₂)]₁₆)CO₂), silver (I) oxide (Ag₂O), silver sulfide (Ag₂S), silver bromide (AgBr), silver iodide (AgI), silver cyanide (AgCN), and combinations thereof. In certain embodiments, the source of phosphate ions is selected from the group consisting of: phosphoric acid (H₃PO₄), ammonium dihydrogen phosphate (NH₄H₂PO₄), sodium dihydrogen phosphate (NaH₂PO₄), lithium dihydrogen phosphate (LiH₂PO₄), potassium dihydrogen phosphate (KH₂PO₄), cesium dihydrogen phosphate (CsH₂PO₄), rubidium dihydrogen phosphate (RbH₂PO₄), and combinations thereof.

Further provided herein is a solution for coating a substrate, the solution comprising a source of magnesium ions, a source of phosphate ions, and a source of silver ions, where the ratio of magnesium ions to phosphate ions in the solution ranges from about 2:1 to about 3:1, and where the pH of the solution ranges from about 5.0 to about 6.9. In certain embodiments, the source of magnesium ions comprises a water-soluble magnesium-containing compound selected from the group consisting of: magnesium nitrate [Mg(NO₃)₂], magnesium chloride (MgCl₂), magnesium sulfate (MgSO₄), magnesium acetate, magnesium benzoate, magnesium bromide (MgBr₂), magnesium chromate, magnesium bromate, magnesium citrate, magnesium formate, magnesium hexafluorosilicate, magnesium iodide (MgI₂), magnesium lactate, magnesium perchlorate, magnesium salicylate, magnesium sulfite, magnesium tartrate, magnesium thiosulfate, hydrates thereof, and combinations thereof. In certain embodiments, the source of silver ions comprises a water-soluble silver compound selected from the group consisting of: silver nitrate (AgNO₃), silver chloride (AgCl), silver fluoride (AgF), silver acetate (AgC₂H₃O₂), silver permanganate (AgMnO₄), silver sulfate (Ag₂SO₄), silver nitrite (AgNO₂), silver bromated (AgBrO₃), silver salicylate (HOC₆H₄COOAg), silver iodate (AgIO₃), silver dichromate (Ag₂Cr₂O₇), silver chromate (Ag₂CrO₄), silver carbonate (Ag₂CO₃), silver citrate (C₆H₈Ag₃O₇), silver phosphate (Ag₃PO₄), silver stearate (Ag[(CH₃)(CH₂)]₁₆)CO₂), silver (I) oxide (Ag₂O), silver sulfide (Ag₂S), silver bromide (AgBr), silver iodide (AgI), silver cyanide (AgCN), and combinations thereof. In certain embodiments, the source of phosphate ions is selected from the group consisting of: phosphoric acid (H₃PO₄), ammonium dihydrogen phosphate (NH₄H₂PO₄), sodium dihydrogen phosphate (NaH₂PO₄), lithium dihydrogen phosphate (LiH₂PO₄), potassium dihydrogen phosphate (KH₂PO₄), cesium dihydrogen phosphate (CsH₂PO₄), rubidium dihydrogen phosphate (RbH₂PO₄), and combinations thereof. In certain embodiments, the pH of the solution is about 5.6.

Further provided herein is a kit for making a bifunctional coating, the kit comprising a first container housing a coating solution comprising silver ions, phosphate ions, and either calcium ions or magnesium ions, and a second container housing a substrate. In certain embodiments, the coating solution comprises both calcium ions and magnesium ions.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the U.S. Patent and Trademark Office upon request and payment of the necessary fees.

FIG. 1: XRD patterns of microwave-processed HA and Ag-doped powders.

FIG. 2: XRD patterns of microwave-processed HA and Ag-doped HA powders in the 2 Theta range of 25°-25°, showing the shift due to Ag doping.

FIG. 3: XRD patterns of coated Ti6Al4V specimens.

FIG. 4: FTIR spectra of coated Ti6Al4V specimens.

FIGS. 5A-5D: SEM images of CaP-0Ag (FIG. 5A), CaP-2Ag (FIG. 5B), CaP-4Ag (FIG. 5C), and CaP-6Ag (FIG. 5D) specimens.

FIGS. 6A-6D: EDS analysis of CaP-0Ag (FIG. 6A), CaP-2Ag (FIG. 6B), CaP-4Ag (FIG. 6C), and CaP-6Ag (FIG. 6D) specimens.

FIG. 7: Water contact angle of uncoated and coated Ti6Al4V specimens.

FIG. 8: ICP results of Ag doped Ti6Al4V specimens.

FIG. 9: Photograph of ZOIs formed by the coated specimens after 24 hours of incubation.

FIG. 10: ZOI coefficient of Ag doped Ti6Al4V specimens.

FIG. 11: The viable number of Colony Forming Units (CFUs) after 24 hours of incubation.

FIG. 12: Photograph of CFUs after 24 hours of incubation.

FIG. 13: MC3T3 cell viability of the control and coated specimens after 4 days of incubation.

FIG. 14: Schematic diagram of the formation mechanism of non-limiting example bifunctional coatings on Ti6Al4V specimens with the application of microwaves.

FIG. 15: XRD patterns of microwave-processed MgP and Ag-doped powders, as well as bare Ti6Al4V.

FIG. 16: Photograph of CFUs after 24 hours of incubation.

### DETAILED DESCRIPTION

Throughout this disclosure, various publications, patents, and published patent specifications are referenced by an identifying citation. The disclosures of these publications, patents, and published patent specifications are hereby incorporated by reference into the present disclosure in their entirety to more fully describe the state of the art to which this invention pertains.

The concept of a “race for the surface”, whereby host and bacterial cells compete in determining the ultimate fate of an implant, indicates that when the host cells colonize the implant surface first, the probability of attachment of bacterial cells becomes low and vice versa. If bacteria are prevented from adhering to the implant surface up front, the risk factor drastically reduces. If the implant surface is capable of rapid osseointegration, then the host bone cells will readily attach to it by the formation of an apatite layer and in the process bacteria cannot adhere to the surface. The situation can be further improved by introducing an antibacterial agent in the coating. Provided herein are bifunctional coatings with antibacterial activity, thus taking advantage of this effect. The bifunctional coatings are prepared by immersing a substrate in a silver-containing solution, and exposing the immersed substrate to microwave radiation. Unlike known silver-containing coatings, the bifunctional coatings described herein have X-ray diffraction patterns which include no peaks attributable to elemental silver.

Being able to mimic natural processes has advantages, such as being benign on any substrate including polymers. However, the conventional biomimetic technique for preparing a coating involves soaking the substrates in simulated body fluids at physiological temperature of 37° C., and constant pH of 7.4, and for about three weeks. Thus, in its purest form, the technique is not industrially viable. Relaxing the stringent biomimetic conditions can accelerate the coating kinetics, thus making this benign process industrially viable as well as versatile. The effect of microwave irradiation on coating solutions enhances the coating kinetics. Pristine Ca—P and Mg-doped Ca—P coatings have been deposited on Ti6Al4V by employing microwave irradiation to substrates immersed in Supersaturated Biomimetic Fluid (SBF). For example, methods of forming hydroxyapatite coatings by microwave irradiation are described in U.S. Patent Application Publication 2014/0308334 A1, which is incorporated herein by reference in its entirety for all purposes. The bifunctional coatings described herein utilize a combination of a modified biomimetic technique with microwave exposure and incorporate silver into the lattice structure for antibacterial activity.

The addition of silver relaxes the stringent condition of biomimicking while still resulting in bioactive as well as antibacterial coatings. The process for preparing these coatings does not damage polymeric substrates. The process is also more benign than biomimmetic coating processes known in the art. Furthermore, this process addresses the needs in the art by providing a “bifunctional” coating which can osseointegrate and be antibacterial at the same time.

The bifunctional coatings herein include a CaP or a MgP. Hydroxyapatite [Ca₁₀(PO₄)₆(OH)₂, “HA”] is a CaP compound that is similar to bone minerals, and has received approval by the FDA for many applications. Other CaP materials include, for example, tricalcium phosphate [TCP, α-TCP, α-Ca₂(PO₄)₂ and β-TCP, β-Ca₃(PO₄)₂], tetracalcium phosphate [TTCP, Ca₄(PO₄)₂O], dicalcium phosphate anhydrous (DCPA, monetite, CaHPO₄), di-calcium phosphate dehydrate (DCPD, brushite, CaHPO₄.2H₂O), and octacalcium phosphate (OCP, Ca₈H₂(PO₄)₆.5H₂O). Though hydroxyapatite is described as an exemplary CaP compound, it is understood that the present disclosure may be modified to create silver-doped CaP compounds other than hydroxyapatite. Similarly, though newberyite (MgHPO₄) and hydrates thereof (e.g., MgHPO₄.3H₂O) are described as exemplary MgP compounds, it is understood that the present disclosure may be modified to create silver-doped MgP compounds other than newberyite. Non-limiting examples of such other MgP compounds include Mg(H₂PO₄)₂ and Mg₃(PO₄)₂.

The deposited coating is composed of a silver-doped CaP, or a silver doped MgP. In some embodiments, the deposited coating is composed of a silver-doped carbonated calcium deficient hydroxyapatite to mimic natural bone, making the coatings not only bioactive but optimally biodegradable. In other embodiments, the deposited coating is composed of a silver-doped newberyite. Regardless of whether the coating is a CaP—Ag or a MgP—Ag coating, the coating releases silver ions over time, resulting in antibacterial activity. Further, at least with respect to CaP—Ag coatings, the coatings may release calcium and phosphate ions from the CaP coating material that can trigger the local formation of bone apatite (a phase belonging to the CaP system) to the implant surface, thus intensifying the bone regeneration around the coated implant.

In some embodiments, the bifunctional coatings are characterized by an X-ray diffraction pattern having no peak that corresponds to elemental silver. Furthermore, the crystal structure of the CaP or MgP is expanded slightly compared to CaP or MgP without silver. This indicates that the silver has been incorporated into the lattice of the CaP or MgP.

The bifunctional coatings can be formed on a wide variety of substrates, such as substrates suitable for orthopedic or dental implants. In some embodiments, the substrate is composed of Ti or a Ti alloy, or other metals such as gold, silver, stainless steel, tantalum, platinum, tungsten, palladium, chromium, cobalt, or alloys, mixtures, composites, and combinations thereof. One non-limiting example substrate material is Ti6Al4V. Other example substrate materials include alumina, zirconia, polyetheretherketone (PEEK), ultra high molecular weight polyethylene (UHMWPE), polymethyl methacrylate (PMMA), polylactic acid (PLA), its co-polymer with glycolic acid (PLGA), polycaprolactone (PCL), polyhydroxyalkanoates (PHA) such as poly-3-hydroxybutyrate (P3HB), or related composites. The implant may further include one or more polycarbonates, polyurethanes, polyesters, perfluorinated hydrocarbons, acrylates, polyamides, epoxy resins, polysiloxanes, or hydrogels.

To make the bifunctional coatings, a substrate is immersed or dipped in, or otherwise exposed to, a coating solution and exposed to microwave radiation. The coating solution is a Supersaturated Biomimetic Fluid (SBF) solution to which silver ions are added. SBF is a supersaturated calcium phosphate or magnesium phosphate solution which does not strictly follow the composition of body fluids. The coating solution generally includes (i) a source of calcium ions or a source of magnesium ions, (ii) a source of phosphate ions, (iii) a source of silver ions, and, optionally, (iv) one or more pH buffers or other additives. Suitable pH buffers include, but are not limited to, sodium bicarbonate, citric acid, acetic acid, or combinations thereof. Suitable additives include, but are not limited to, silica, such as nanocrystalline and/or colloidal silica.

The source calcium ions can be any soluble Ca-containing compound. Non-limiting examples include calcium nitrate [Ca(NO₃)₂], calcium chloride (CaCl₂), calcium bromide (CaBr₂), calcium acetate [(CH₃COO)₂Ca], calcium citrate, calcium iodide (CaI₂), calcium lactate, calcium gluconate, calcium fumarate, calcium oxide (CaO), calcium hydroxide [Ca(OH)₂], calcium benzoate, calcium formate, calcium butyrate, calcium isobutyrate, calcium malate, calcium maleate, calcium propionate, calcium valerate, combinations thereof, or hydrates thereof such as calcium nitrate tetrahydrate [Ca(NO₃)₂.4H₂O], calcium citrate tetrahydrate, or calcium bromide hexahydrate.

The source of magnesium ions can be any soluble Mg-containing compound. Non-limiting examples include magnesium nitrate [Mg(NO₃)₂], magnesium chloride (MgCl₂), magnesium sulfate (MgSO₄), magnesium acetate, magnesium benzoate, magnesium bromide (MgBr₂), magnesium chromate, magnesium bromate, magnesium citrate, magnesium formate, magnesium hexafluorosilicate, magnesium iodide (MgI₂), magnesium lactate, magnesium perchlorate, magnesium salicylate, magnesium sulfite, magnesium tartrate, magnesium thiosulfate, combinations thereof, or hydrates thereof such as magnesium nitrate hexahydrate [Mg(NO₃)₂.6H₂O], magnesium iodide hexahydrate, or magnesium iodide octahydrate.

The source of phosphate ions can be any compound which dissociates in solution to create dihydrogen phosphate (H₂PO₄⁻) ions, hydrogen phosphate (HPO₄²⁻) ions, or phosphate (PO₄³⁻) ions. Non-limiting examples include phosphoric acid (H₃PO₄); ammonium dihydrogen phosphate (NH₄H₂PO₄); Group I salts of dihydrogen phosphate, including sodium dihydrogen phosphate (NaH₂PO₄), lithium dihydrogen phosphate (LiH₂PO₄), potassium dihydrogen phosphate (KH₂PO₄), cesium dihydrogen phosphate (CsH₂PO₄), and rubidium dihydrogen phosphate (RbH₂PO₄); and combinations thereof.

For CaP—Ag coatings, the coating solution should have a ratio of calcium ions to phosphate ions present in the solution (i.e., the Ca/P ratio) of about 1.67 to ensure formation of hydroxyapatite. The Ca/P ratio may generally range from about 1.5 to about 1.7, but is optimally about 1.67. The pH of the coating solution for CaP—Ag coatings is not particularly critical. In one non-limiting example, the pH of the coating solution is about 7.4.

For MgP—Ag coatings, the coating solution should have a ratio of magnesium ions to phosphate ions present in the coating solution (i.e., the Mg/P ratio) ranging from about 2:1 to about 3:1 to ensure formation of newberyite. Furthermore, for MgP—Ag coatings, the pH of the coating solution should be slightly acidic to ensure formation of newberyite. In one non-limiting example, the pH of the coating solution is about 5.6. However, the pH of the coating solution for MgP—Ag coatings may range from about 5.0 to about 6.9.

Silver ions are added to the coating solution in order to incorporate silver into the coating. The silver ions can be added to the coating solution by adding one or more soluble silver compounds to the coating solution. Non-limiting examples of soluble silver compounds are silver nitrate (AgNO₃), silver chloride (AgCl), silver fluoride (AgF), silver acetate (AgC₂H₃O₂), silver permanganate (AgMnO₄), silver sulfate (Ag₂SO₄), silver nitrite (AgNO₂), silver bromate (AgBrO₃), silver salicylate (HOC₆H₄COOAg), silver iodate (AgIO₃), silver dichromate (Ag₂Cr₂O₇), silver chromate (Ag₂CrO₄), silver carbonate (Ag₂CO₃), silver citrate (C₆H₈Ag₃O₇), silver phosphate (Ag₃PO₄), silver stearate (Ag[(CH₃)(CH₂)]₁₆)CO₂), silver (I) oxide (Ag₂O), silver sulfide (Ag₂S), silver bromide (AgBr), silver iodide (AgI), and silver cyanide (AgCN).

The silver ions can be added in an amount ranging from about 0.01% to about 10% by weight of the silver compound relative to weight of the Ca-containing or Mg-containing compound in the coating solution (e.g., relative to the weight of Ca(NO₃)₂ for CaP—Ag coatings, or relative to the weight of Mg(NO₃)₂ for MgP—Ag coatings). The greater the amount of silver added to the coating solution, the longer the constant release of the silver from the bifunctional coating. Thus, the duration of silver release from the bifunctional coating can be customized and controlled.

It should be noted that for ease of explanation, the coating solution is described herein as including either calcium ions or magnesium ions, but the present disclosure nonetheless encompasses embodiments where the coating solution includes both magnesium ions and calcium ions, in addition to the silver ions. In such cases, the amount of silver ions in the coating solution is relative to the combined amounts of calcium ions and magnesiums ions, and the resulting coating is generally a silver-doped Ca—Mg—P phase (which may or may not further include various dopans such as C1 atoms, depending on the composition of the coating solution). Furthermore, the coating solution may also include a soluble calcium phosphate as a source of both calcium ions and phosphate ions, and/or a soluble magnesium phosphate as a source of both magnesium ions and phosphate ions.

Non-limiting example coating solutions for producing CaP—Ag coatings have the following formulation: 0.6612 g Ca(NO₃)₂.H₂O, 0.2016 g NaH₂PO₄, 0.0672 g NaHCO₃, and from 0.0132 g to 0.0397 g AgNO₃, all in 200 mL H₂O. In these coating solutions, Ca(NO₃)₂.H₂O serves as the source of calcium ions, NaH₂PO₄ serves as the source of phosphate ions, NaHCO₃ is a pH buffer, and AgNO₃ serves as the source of silver ions.

Non-limiting example coating solutions for producing MgP—Ag coatings have the following formulation: 0.6612 g Mg(NO₃)₂.6H₂O, 0.2016 g NaH₂PO₄, 0.0672 g NaHCO₃, and from 0.0132 g to 0.0397 g AgNO₃, all in 200 mL H₂O. In these coating solutions, Mg(NO₃)₂.6H₂O serves as the source of magnesium ions, NaH₂PO₄ serves as the source of phosphate ions, NaHCO₃ is a pH buffer, and AgNO₃ serves as the source of silver ions.

Non-limiting example coating solutions for producing silver-doped Ca—Mg—P coatings have the following formulation: 0.2267 g NaHCO₃, 0.11025 g CaCl₂.2H₂O, 0.1525 g MgCl₂.6H₂O, 0.1361 g KH₂PO₄, and from 0.0132 g to 0.0397 g AgNO₃, all in 200 mL H₂O. In these coating solutions, CaCl₂.2H₂O serves as the source of calcium ions, MgCl₂.6H₂O serves as the source of magnesium ions, AgNO₃ serves as the source of silver ions, KH₂PO₄ serves as the source of phosphate ions, and NaHCO₃ is a pH buffer.

The substrate is immersed in the coating solution containing the silver ions, and then exposed to microwave radiation for a period of time sufficient to form a silver-doped coating, where the coating is composed of CaP, MgP, or a combination of CaP and MgP. Though irradiating the substrate with microwaves while immersed in the coating solution is described, irradiating the substrate following removal of the substrate from the coating solution is nonetheless encompassed within the scope of the present disclosure.

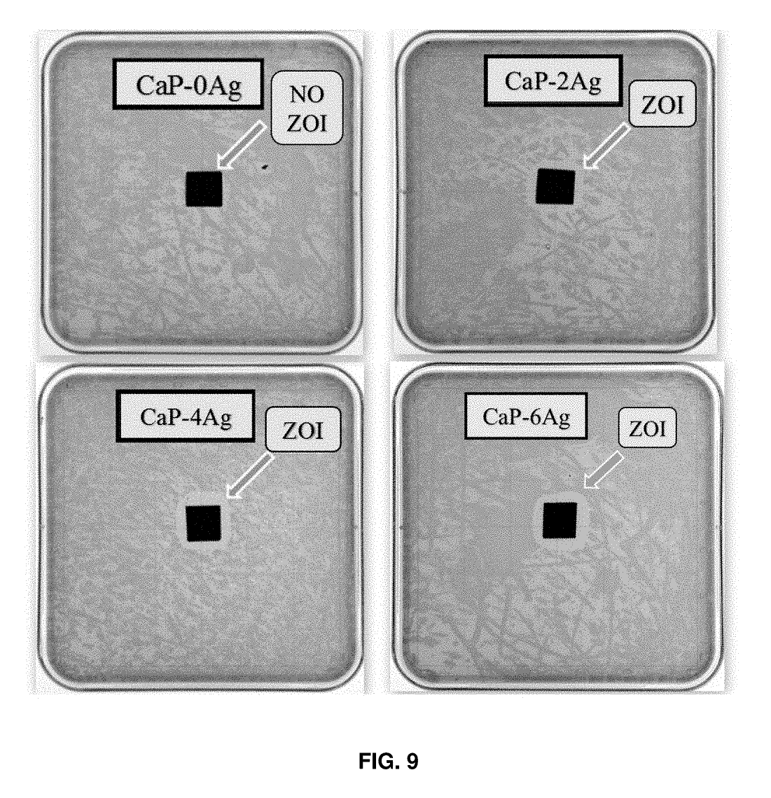

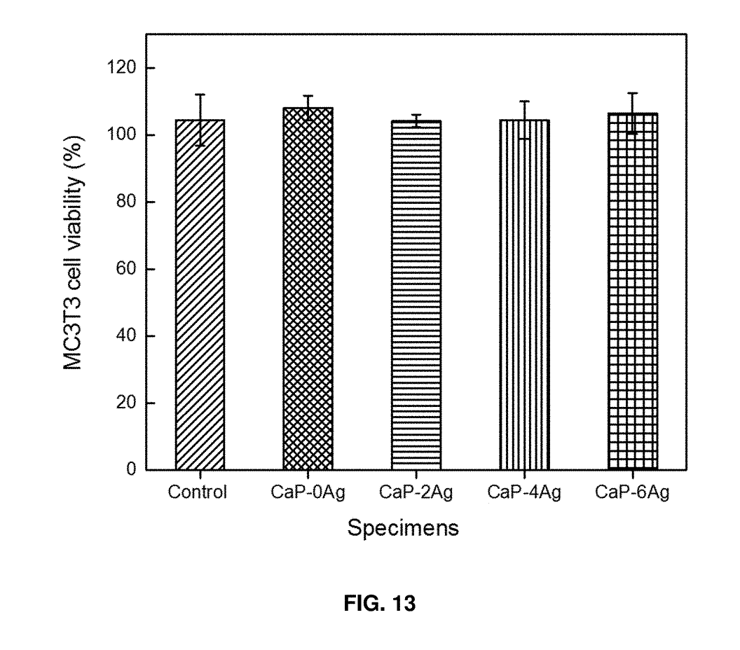

The microwave irradiation can last for about 1 hours or less, or about 45 minutes or less, or about 30 minutes or less. In certain examples, the microwave irradiation lasts for about 10 minutes or less. For CaP—Ag coatings, to ensure hydroxyapatite formation, the coating solution should have a Ca/P ratio of about 1.67, and the microwave irradiation of the immersed substrate should be 8-10 minutes at a microwave power level of about 1200 W. For MgP—Ag coatings, to ensure newberyite formation, the coating solution should have a Mg/P ratio ranging from about 2:1 to about 3:1, and the microwave irradiation of the immersed substrate should be about 10 minutes at a microwave power of about 1200 W. In certain embodiments, the resulting bifunctional coating applied to the substrate has an overall thickness of about 100-900 nm to about 1-10 μm. In some embodiments, the bifunctional coating applied to the substrate has an overall thickness of about 15 μm, about 10 μm, or about 5 μm. Regardless of the bifunctional coating's thickness, silver ions are released over time from the bifunctional coating, resulting in antibacterial activity, as seen from FIGS. 9-12, 16. Furthermore, as seen from FIG. 13, the bifunctional coating is not cytotoxic. Thus, the bifunctional coating is advantageously used for orthopedic or dental implants.

It is further envisioned that the compositions and methods described herein can be embodied in the form of a kit or kits. A non-limiting example of such a kit is a kit for preparing a bifunctional coating, the kit comprising a substrate and a coating solution containing silver ions in separate containers, where the containers may or may not be present in a combined configuration. Many other kits are possible, such as kits for that further comprise an additive such as silica. Also provided are kits for preparing a coating solution, where such kits contain sources of calcium and/or magnesium ions, phosphate ions, and silver ions in two or more containers. The kits may further include instructions for using the components of the kit to practice the subject methods. The instructions for practicing the subject methods are generally recorded on a suitable recording medium. For example, the instructions may be present in the kits as a package insert or in the labeling of the container of the kit or components thereof. In other embodiments, the instructions are present as an electronic storage data file present on a suitable computer readable storage medium, such as a flash drive or CD-ROM. In other embodiments, the actual instructions are not present in the kit, but means for obtaining the instructions from a remote source, such as via the internet, are provided. An example of this embodiment is a kit that includes a web address where the instructions can be viewed and/or from which the instructions can be downloaded. As with the instructions, this means for obtaining the instructions is recorded on a suitable substrate.

### EXAMPLES

As most orthopedic and dental implants in the biomedical industry are made of Ti6Al4V, this was selected as a substrate for example purposes. However, other substrates are possible and entirely encompassed within the scope of the present disclosure. As described in these Examples, the morphologies and compositions of the coatings were characterized using X-rays Diffraction analysis (XRD), Fourier Transform Infrared Spectroscopy (FTIR), Scanning Electron Microscopy (SEM), and in-vitro antibacterial test assays. Keeping in mind the toxic effect of Ag, cytocompatibility tests of the microwave coated Ti substrates were also assessed in vitro.

### Example I—Calcium Phosphate Doped with Silver

This example describes the development of bifunctional coatings on Ti6Al4V alloys using a microwave assisted modified biomimetic coating process. These bifunctional coatings, comprising calcium deficient hydroxyapatite, render the metallic substrates osseointegrable while simultaneously providing resistance against bacterial infections. Enhanced osteoblast attachments to the substrate also prevent bacterial attachment. The antibacterial properties of the coatings are further boosted by incorporating Ag⁺ ions, an antibacterial agent. The process synergistically applies beneficial aspects of supersaturated fluids as used in biomimetic coatings along with microwave radiation. The process kinetics are greatly accelerated by relaxing the stringent requirements of biomimetics along with the application of microwave irradiation. This Example includes physical evaluations of such coatings, including X-ray Diffraction (XRD) analysis, Fourier Transform Infrared (FTIR) spectroscopy, contact angle measurements, Scanning Electron Microscopy (SEM), and also a determination of the release profile of Ag⁺ ions from the coatings.

To evaluate the dissolution of Ag⁺ into the HA lattice, powders were also synthesized during the coating process. The Whole Pattern Fitting (WPF) and Rietveld refinement of the X-ray diffraction patterns of the synthesized powders confirmed the incorporation of Ag⁺ ions into the HA lattice. In-vitro test assays such as zone of inhibition (ZOI) tests, and counting of colony forming units (CFUs), were used in evaluating the antibacterial characteristics of the coatings against E. coli. The results show a sustained release of Ag⁺ ions from the respective coatings, and its correlation with the area of the ZOIs. The cytocompatibility tests confirmed an enhanced cell viability on all the coated Ti6Al4V specimens, emphasizing the fact that the released amount of Ag⁺ ions did not introduce any cytotoxicity.

Material Preparation

A 10 cm×10 cm Ti alloy (Ti6Al4V) plate was cut into 1 cm×1 cm squares. Sample surfaces were sequentially polished with SiC papers, progressing to finer grit sizes. They were then ultrasonically rinsed in isopropyl alcohol and distilled water for 10 mins respectively. After the cleaning process, the samples were dried at 60° C. for 30 min and cooled to room temperature.

Pre-Treatment

In order to ensure proper attachment of functional groups on the Ti alloy substrates, the squares were etched in 10 mM NaOH at 70° C. for 24 hours. Afterwards, all samples were cleaned by rinsing them in distilled water. After cleaning, the samples were dried at 60° C. for 1 hr and then cooled to room temperature. Finally, the NaOH etched samples were heated at 550° C. in an oven furnace for a period of 1 h, and allowed to cool to room temperature at the programmed rate of the furnace.

Coating and Powder Preparation

The coating solutions with Ag⁺ ions were prepared as per the recipe shown in Table 2.

TABLE 2
Composition of coating solutions

| | | | | AgNO₃ | | |
|---|---|---|---|---|---|---|
| H₂O | Ca(NO₃)₂•4H₂O | NaH₂PO₄ | NaHCO₃ | Wt. (gms) | | Wt. % |
| 200 ml | 0.6612 gms | 0.2016 | 0.0672 | 0 | gm | (0% |
| | | gms | gms | | | by wt.) |
| 200 ml | 0.6478 gms | 0.2016 | 0.0672 | 0.0132 | gms | (2% |
| | | gms | gms | | | by wt.) |
| 200 ml | 0.6347 gms | 0.2016 | 0.0672 | 0.0264 | gms | (4% |
| | | gms | gms | | | by wt.) |
| 200 ml | 0.6215 gms | 0.2016 | 0.0672 | 0.0397 | gms | (6% |
| | | gms | gms | | | by wt.) |

AgNO₃ was used as the source of the Ag⁺ ions. Three different weight percentages of AgNO₃: 6, 4, 2 wt. % were employed. (These weight percentages are with respect to the weight of Ca(NO₃)₂.4H₂O.) The control group used for this experiment was Ti alloy coated with only CaP, i.e., without any inclusion of Ag⁺ ions. Four samples per each Ag content were prepared. The etched samples were placed in one 200 ml beaker, filled with 100 ml coating solution. The top of the beaker was covered by a larger beaker to avoid spilling. The set-up was then put inside a 1200 W microwave oven (Panasonic) and irradiated at the highest power for 4 mins. The above steps were repeated once more to ensure uniformity of coatings. The coated substrates were also dried at room temperature. The precipitates were also synthesized without the presence of substrates and were collected in 50 ml centrifuge tubes. Further, they were centrifuged and dried in an air oven furnace at 60° C. for 1 hr.

TABLE 3
Crystal size of the microwave-processed HA and
Ag-doped HA powders

| Serial No. | Specimen Name | Crystallite size (nm) |
|---|---|---|
| 1. | CaP-0Ag | 40.4 |
| 2. | CaP-2Ag | 27 |
| 3. | CaP-4Ag | 25.8 |
| 4. | CaP-6Ag | 25.1 |

Powder and Coating Characterization

The precipitates collected during the coating process were centrifuged and dried for further characterization by XRD. The phase compositions of the as-synthesized powders and as-coated samples were identified by XRD (Ultima III, Rigaku) with mono-chromated Cu Kα radiation (44 KV, 40 mA) over a 2θ range of 10-60°. A whole pattern fitting (WPF) analysis and Rietveld refinement were conducted using the MDI Jade software (Materials Data Inc., Livermore, Calif.) to determine the crystallite size and lattice parameters of the microwave processed HA and Ag-HA powders. Surface morphologies and elemental compositions of the coated samples were analyzed using SEM (FEI Quanta 3D FEG) equipped with an energy dispersive X-ray spectroscopy (EDS, Oxford INCA). Elemental analyses were performed at 20 KV with a working distance of 10 mm. Functional groups present in the coatings were characterized by FTIR (FTIR, UMA-600 Microscope, Varian Excalibur Series) using an ATR diamond crystal for 256 scans in the range between 4000 and 700 cm⁻¹ with a resolution of 1 cm⁻¹.

Contact Angle Measurements

Contact angle measurements were performed by a contact angle meter (Model CAM-MTCRO, Tantec). Water droplets were carefully placed on the surface of uncoated Ti6Al4V substrates and also on all the coated specimens for contact angle measurement.

Ag⁺ Ion Release

In order to examine the kinetics of Ag⁺ ion release, the various CaP—Ag coated samples were immersed in 10 ml of ultra-pure water over a period of 6 days. The ultra-pure water was renewed every day and the Ag⁺ ion concentration was checked employing an inductive coupled plasma mass spectrometer (Thermo Scientific XSeries 2 ICPMS).

In-Vitro Antibacterial Test

To confirm the antibacterial functionality, three test assays were carried out. E. coli (W3110) was used to evaluate the effectiveness of the as-developed antibacterial coatings. Super Broth (SB) was used as the growth medium. The overnight culture of W31101 E. coli bacteria was established and used in all the in-vitro antibacterial test assays.

Zone of Inhibition (ZOI) Method

To assess the antibacterial activity distal to the surface, the traditional ZOI tests were carried out on agar plates. Ag⁺-doped coated implants along with the control were placed on individual agar plates and each of them were exposed to 100 μl of bacterial suspension (OD600=2.0). The idea behind using a higher concentration was to ensure the formation of a spread bacterial colony (lawn) on the agar plate surface. To ensure uniform spread, circular beads were used. Finally, the plates containing the coupons were kept in an incubator initially for 24 hrs at 37° C. The formation of ZOI was then inspected and measured using Vernier calipers. Further, they were incubated for 6 more days. For easy understanding, ZOI was quantified by a simple coefficient method. The following formula was used:

ZOI coefficient=Total ZOI area/Coupon area

The coefficient was calculated rounding to 2 decimal places. Obtaining a numerical value greater than 1 states the antibacterial functionality of the coupon. Repeated measurements of ZOIs were obtained after incubating the coupons for the next 2, 4, 6 days.

Plate Counting Method—Colony Forming Units

To quantify the antibacterial properties of coated Ti samples, the plate counting method was employed wherein the colony forming units (CFUs) were counted and compared. The coated specimens were placed in individual 10 ml sterile test tubes and exposed to 2 ml of bacterial suspension diluted with SB (OD600=0.2). The test tubes were then placed inside an incubator at 37° C. for 3 hrs. At the end of the incubation period, the samples were removed and 100 μl from each test tube were plated out on solid agar plates and incubated overnight at 37° C. The next day, the CFUs on each plate were counted and compared.

Cytotoxicity Tests

All the coated samples were prepared, autoclaved and incubated in minimum essential medium alpha (MEM-α Thermo Scientific, Logan, Utah, USA), supplemented with 10% fetal bovine serum (FBS, Thermo Scientific HyClone) and 1% penicillin/streptomycin (0.2 g/ml) at 5% CO₂ and 37° C. for 24 h. The extracts were then collected and used to culture MC3T3 cells (CRL-2593™, ATCC, Manassas, Va., USA). The extracts were initially seeded at 70% confluency and after 4 days the cell viability was measured. To assess the cell viability, the cells were treated with thiazolyl blue tetrazolium bromide (MTT, Sigma-Aldrich, St. Louis, Mo., USA) for 4 h, insoluble formazan dissolved in DMSO, and the optical density was measured. The % cell viability was calculated and expressed as a measure of cytotoxicity and compared to untreated controls.

Statistical Analysis

All the tests were performed in triplicates. The results are represented in means±SD. One-way analysis of variance (one way-ANOVA) with Tukey test was conducted to determine the statistical difference between groups and (p<0.05) was considered significant.

Results

Powder and Coating Characterization

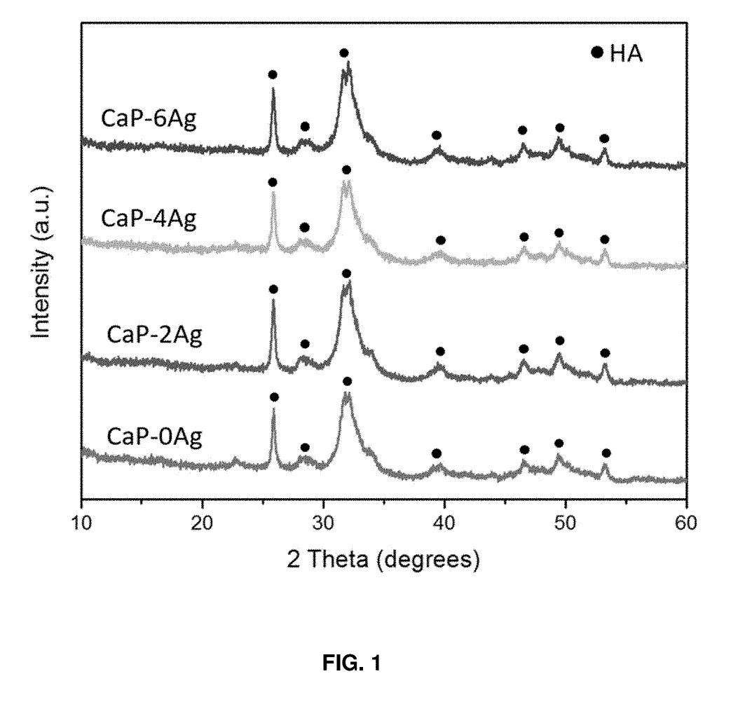

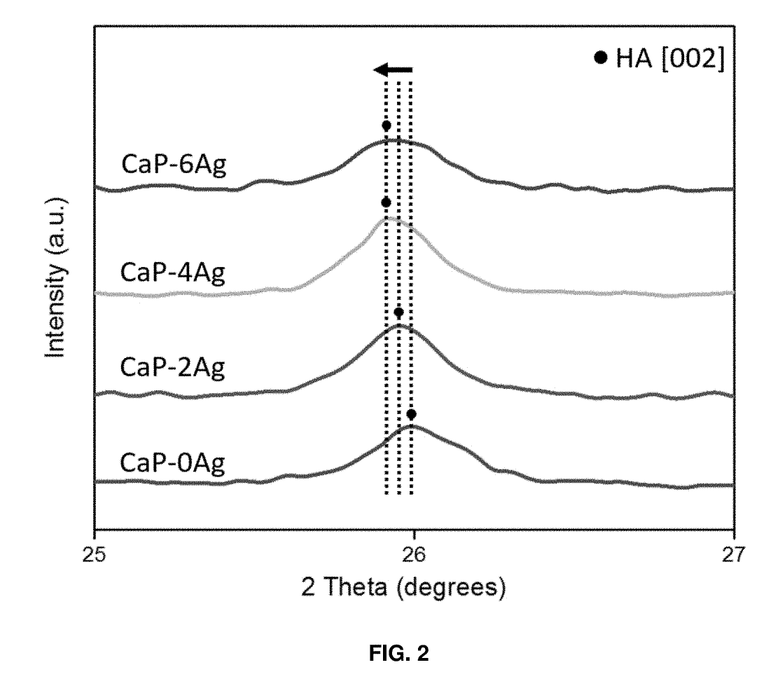

FIG. 1 shows the XRD patterns of the microwave processed HA and Ag⁺-doped HA powders. The peak broadening effect is quite evident from the patterns. All the diffraction patterns were almost similar and matched with the hexagonal HA crystal (PDF No. 97-015-7481) with hP63/m as the space group. A slight shift in the diffraction patterns were noted in the case of the Ag doped samples. FIG. 2 shows the pattern diffraction specifically over the 2θ angles 25°-27° for easy visual of the shift. The crystallite sizes of the synthesized powders are presented in Table 3. All the crystal size as calculated by the Jade software (MDI 2010) software were in the lower nanometer range and a minute decrease was also reported with the increase in Ag doping. The unit cell parameters as calculated by the WPF analysis and Rietveld refinement are tabulated in Table 4. When compared with the lattice parameters of pristine HA, a change can be seen in case of Ag⁺-doped HA powders. The refined crystal lattice parameters a=b and c have linearly increased with increasing substitution level. This change in the unit cell parameters indicates a lattice distortion.

TABLE 4
Unit cell parameters of the microwave-processed
HA and Ag-doped HA powders

| Serial | | | |
|---|---|---|---|
| No. | Specimen Name | a = b (Angstrom) | c (Angstrom) |
| 1. | CaP-0Ag | 9.45351 | 6.87282 |
| 2. | CaP-2Ag | 9.46081 | 6.87889 |
| 3. | CaP-4Ag | 9.46369 | 6.87973 |
| 4. | CaP-6Ag | 9.47301 | 6.88369 |

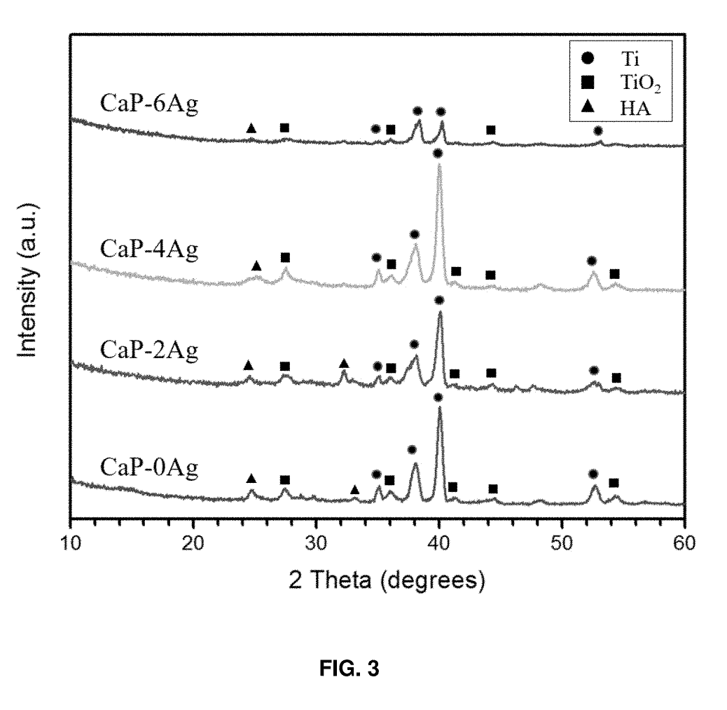

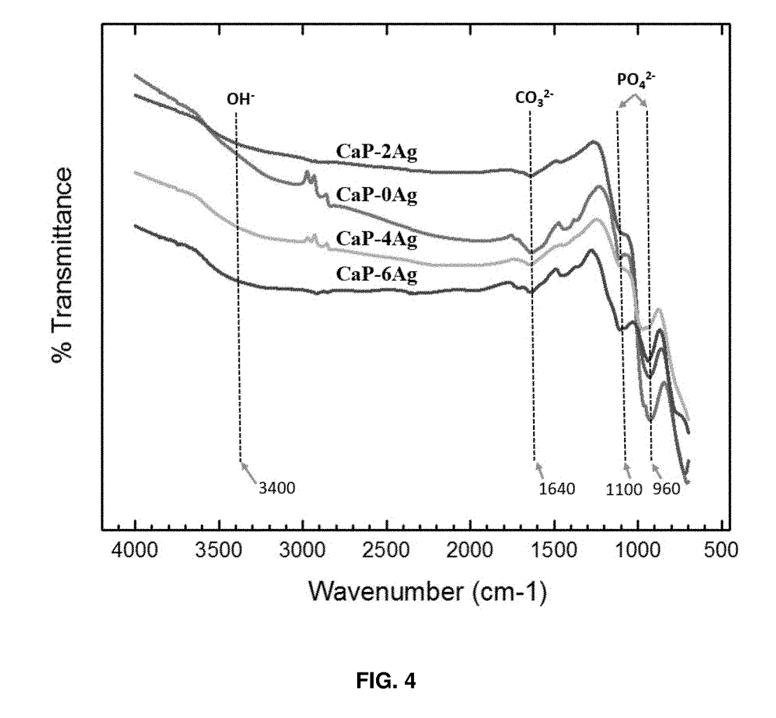

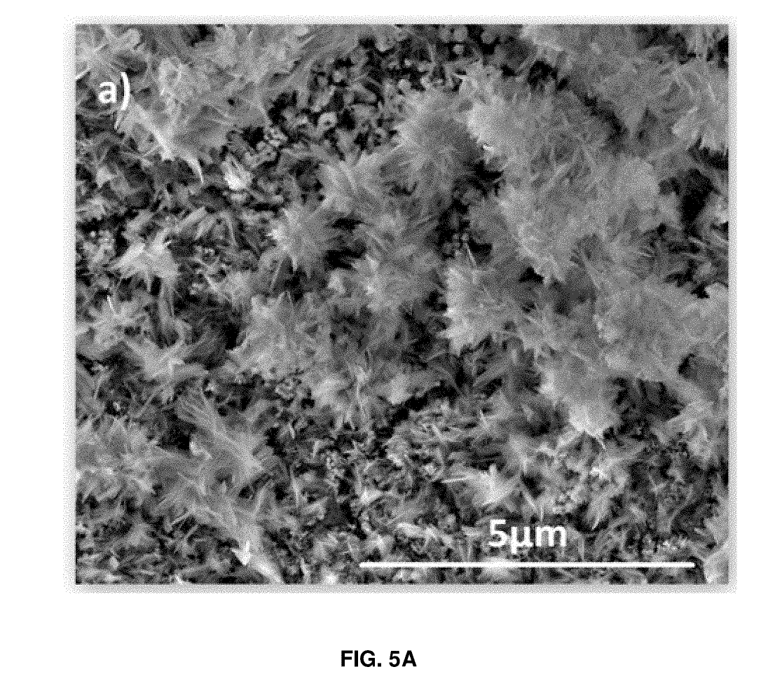

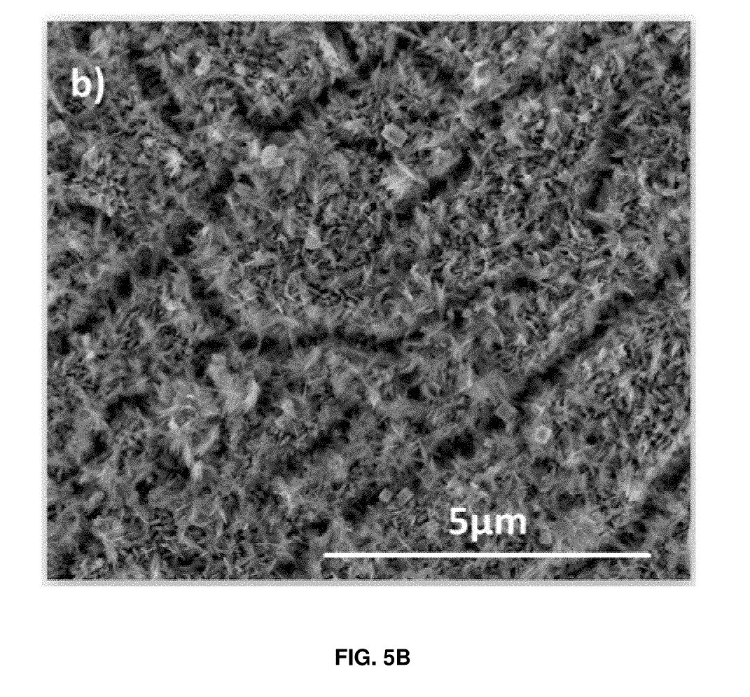

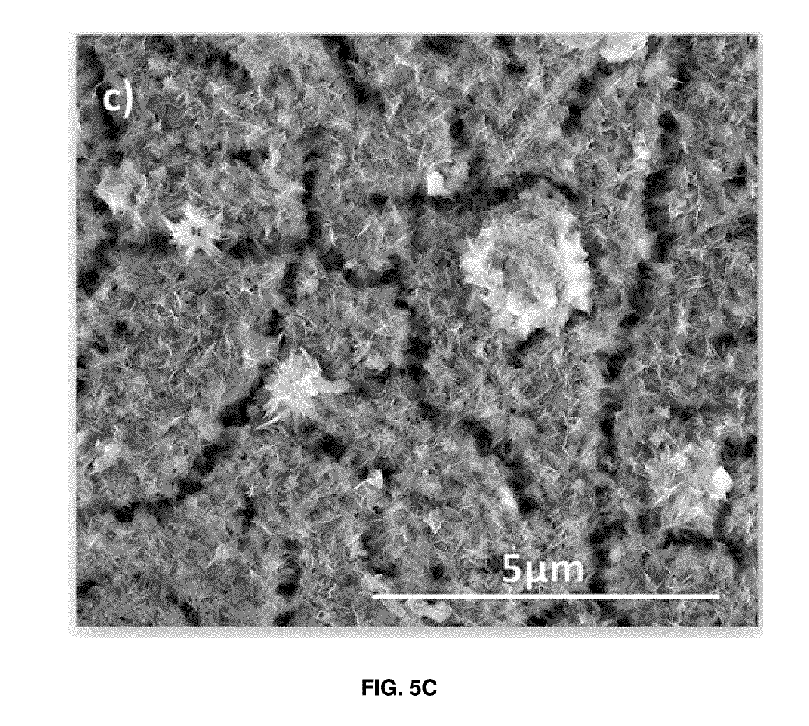

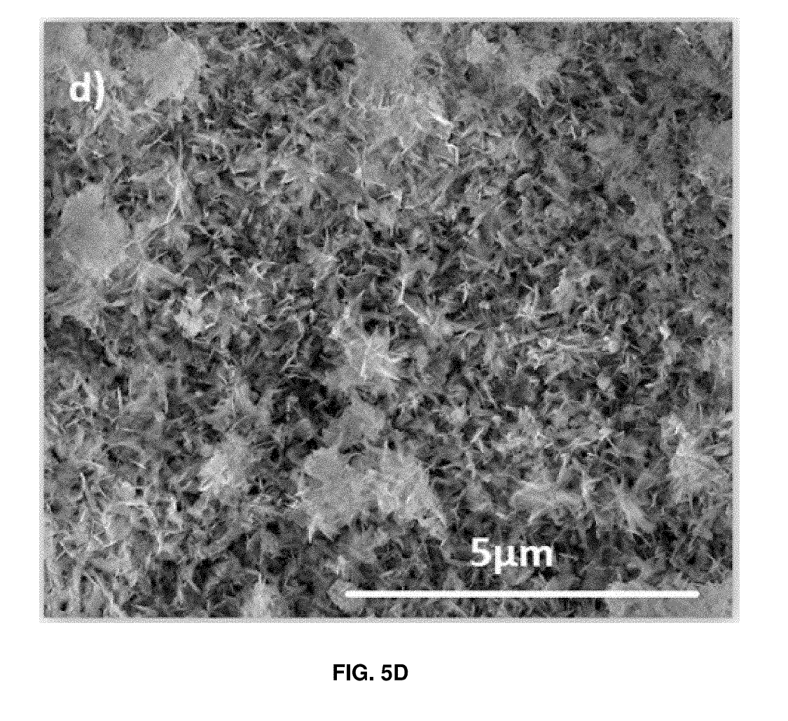

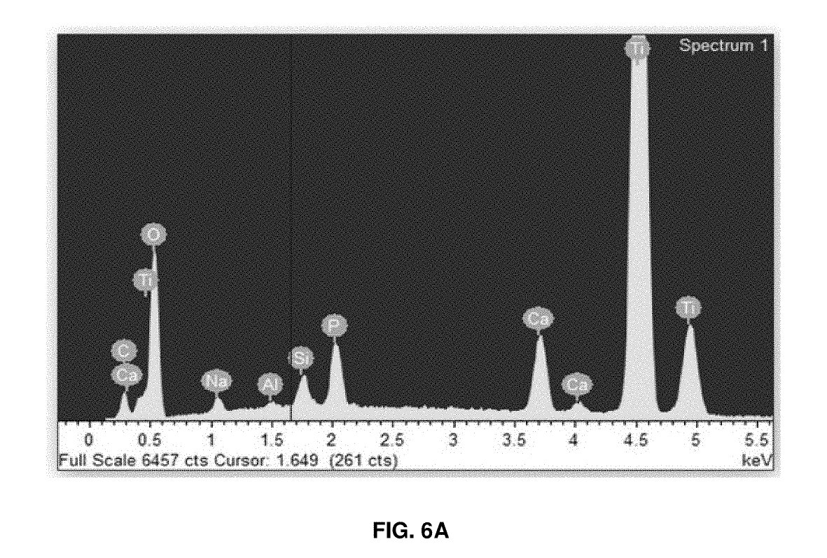

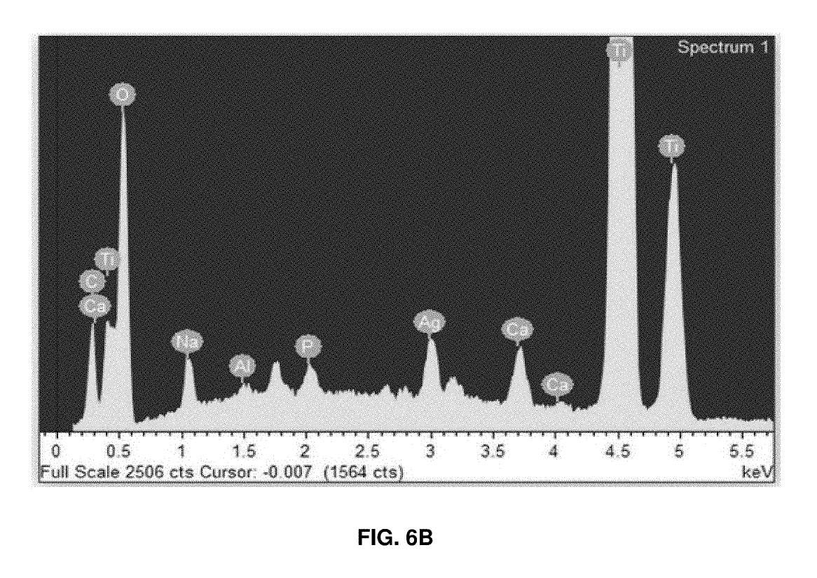

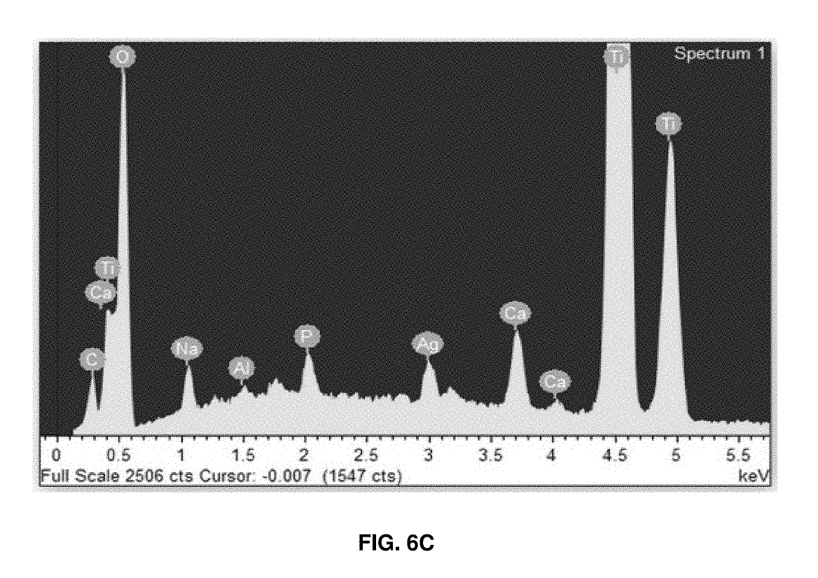

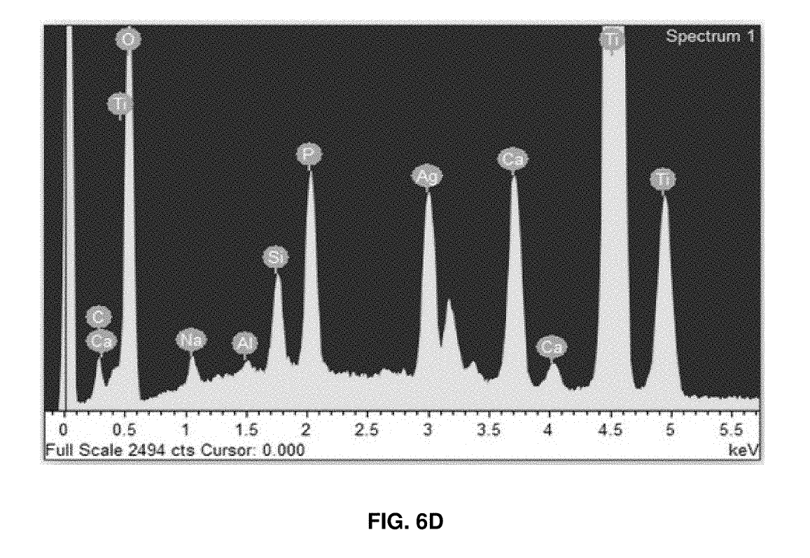

FIG. 3 shows the X-ray diffraction pattern of the coated specimens. Heat treatment resulted in the formation of TiO₂ and the phase was identified as rutile (PDF No. 97-000-9161) with a tetragonal crystal system. The intensities of the diffraction peaks of rutile (TiO₂) appeared quite similar in case of all the samples. Microwave assisted coating on Ti6Al4V resulted in the formation of new peaks which corresponded to HA (PDF No. 09-432). Most of the HA peaks formed after microwave processing were nanocrystalline, which is indicated by line broadening in the XRD patterns. However, diffraction peaks along the [002] plane exhibited relatively higher intensity. This signifies that the preferred orientation for HA growth is along this specific plane, (002)_{HA}. In the case of Ag doped samples, XRD could not successfully detect peaks of elemental Ag due to its minimal concentration. The FTIR spectra of the coated substrates are displayed in FIG. 4. The hydroxyl (OH⁻) group stretching is detected at 3400 cm⁻¹ which proves the presence of HA. However, it lies amidst the broad bands of absorbed water (2600 cm⁻¹-3600 cm⁻¹). Carbonate (CO₃²⁻) group, recognized at 1640 cm⁻¹, showed visible peaks. Biomimetic HA is calcium deficient and it is mostly substituted by a carbonate group. In this case corresponding to the absorption band, PO₄³⁻ substitutes a tetrahedral group with CO₃²⁻. Peaks and shoulders at 1100 cm⁻¹ and 960 cm⁻¹ correspond to the phosphate (PO₄²⁻) group due to the v1 and stretching v3 vibration mode. FIGS. 5A-5D show the SEM images of the coated specimens. The images confirm the uniformity of HA coatings and all the samples are densely covered with flake-like crystals. The doping of Ag did not affect the coating homogeneity. The EDS results presented in FIGS. 6A-6D clearly indicate the presence of Ag along with Ca and P in the coatings.

Contact Angle

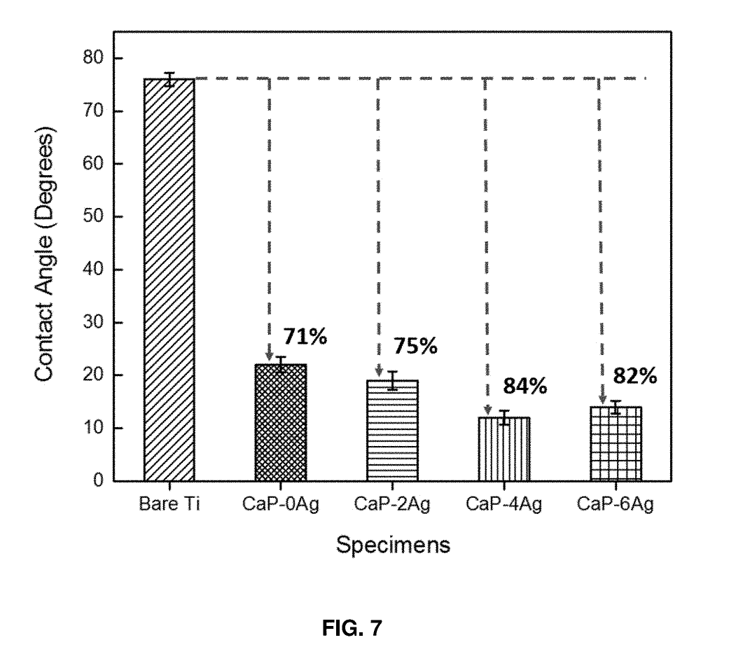

FIG. 7 shows a comparison of the water contact angle measurements of various specimens. In this case, bare Ti was used as a control in order to compare the extent of hydrophilicity between coated and uncoated Ti substrate. The difference is quite significant in the case of the coated samples (decrease in the range of 71%-82%), which strongly indicates the hydrophilic nature of HA. The water contact angle measured for bare Ti is 76°, while all the HA coated samples were in the range of 20°. Doping of the various wt. % of Ag did not influence the wettability of the substrate surfaces and even no specific decreasing trend was noticed.

Ag⁺ Ion Release Behavior

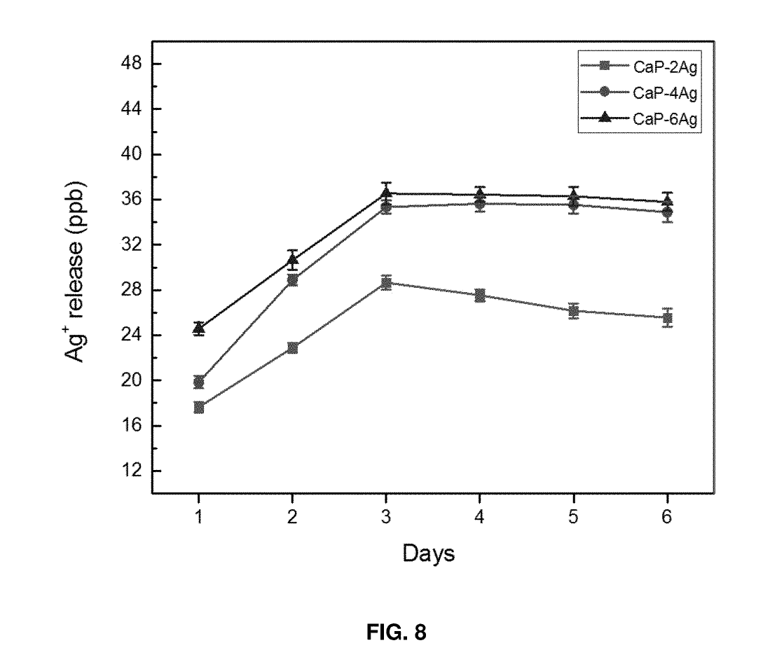

The Ag⁺ ion release characteristics of the coated Ti substrates are displayed in FIG. 8. The graph shows a measure of the ion release in parts per billion (ppb) over a period of 6 days. The standard deviation amongst the data is considerably less for the CaP-2Ag samples because of the minute release of Ag⁺ ions. Coatings which contained the highest percentage of Ag (CaP-6Ag) released the maximum amount of ions. The Ag⁺ release trend for all the coatings can be interpreted as follows: In case of all the coated samples, the release was quite high in the first 3 days. The coatings which contained 6 wt. % and 4 wt. % Ag experienced a gradual decrease in the release of Ag⁺ ions, while the decrease was comparatively steep in case of coatings which contained 2 wt. % Ag. The ion release trends of CaP-4Ag and CaP-6Ag samples highlight the sustainability of the coatings to inhibit bacterial infections days after the actual implantation. In all cases of coated Ti6Al4V samples, the ion release concentration was much less than the cytotoxic concentration of 1600 ppb for somatic.

Antibacterial Functionality

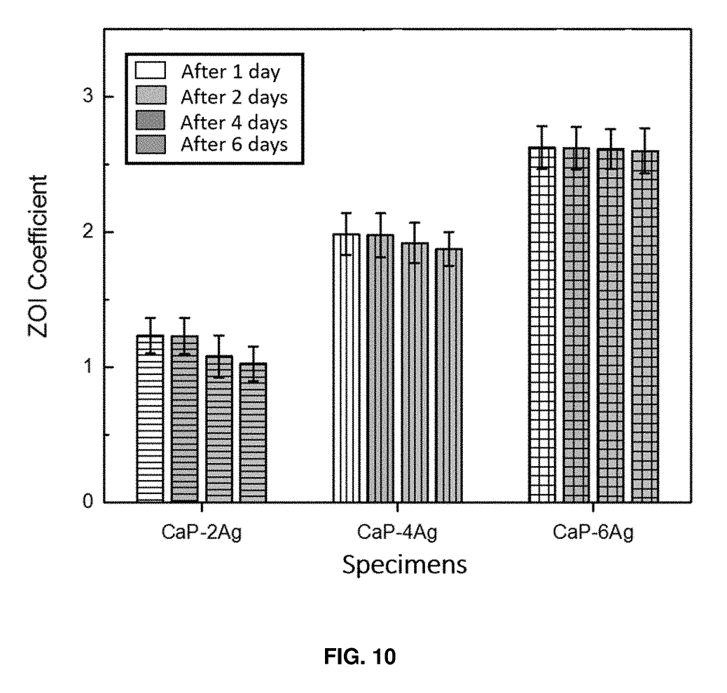

The ZOI test on solid agar plates provided a comprehensive analysis of the antibacterial functionality of coated Ti alloys. No proper ZOI was observed in case of CaP-0Ag samples (FIG. 9A). This indicates that HA coated Ti6Al4V samples do not possess the capability to resist bacterial growth distal to the surface. All other HA coated samples doped with Ag exhibited distinct ZOIs, though the individual extent varied with the varying compositions of Ag. Sample coatings containing as low as 2 wt. % Ag successfully produced a distinct ZOI. FIGS. 9B-9D display the ZOIs produced by different samples on solid agar plates after incubation of 24 hrs. As seen from the images, the largest ZOI was produced by CaP-6Ag, which contained the highest amounts of Ag in this experiment. In order to quantify and compare the areas of different ZOIs, the ZOI coefficients were calculated and presented in FIG. 10. Measurements of ZOIs were recorded after 2, 4, and 6 days in order to check on its enduring resistance against bacterial growth. A coefficient-value of more than 1 confirms the antibacterial functionality of the specimens. As seen in FIG. 10, the ZOI coefficient increases with an increase in Ag concentration in the coatings, indicating enhanced bacterial resistance. The highest ZOI coefficient calculated was 2.625, for CaP-6Ag, while the lowest was 1.232, for CaP-2Ag, after 24 hrs of incubation. However, in the case of the CaP-2Ag samples, the ZOI was distinct enough even after 6 days of bacterial exposure (ZOI Coeff.=1.025). This means that in spite of low Ag concentration, it still had the capability to resist bacterial growth around it over the period. For both CaP-2Ag and CaP-4Ag samples, the ZOI coefficient decreased ˜16% and ˜5%, respectively, over 6 days. But for CaP-6Ag, the decrease was quite negligible (˜0.5%), highlighting its strong endurance against bacterial growth over days.

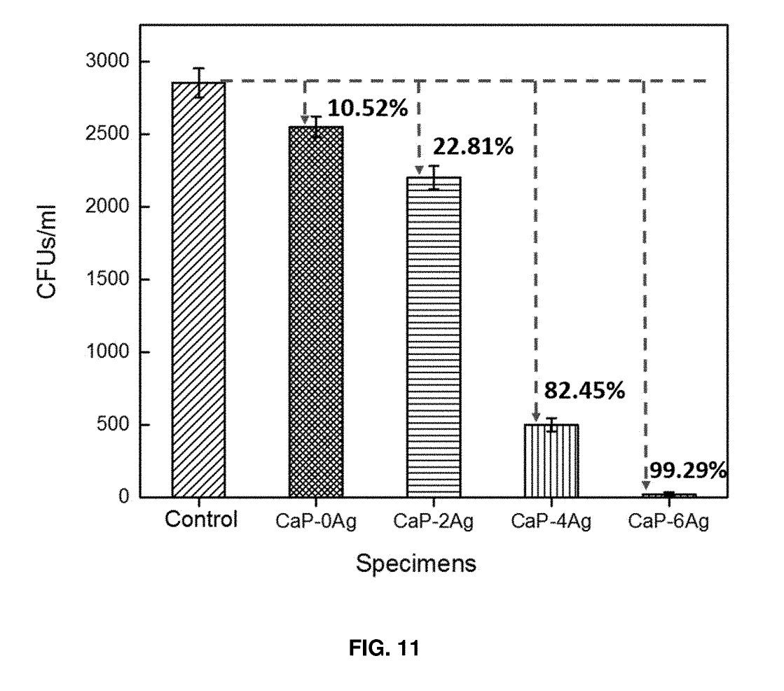

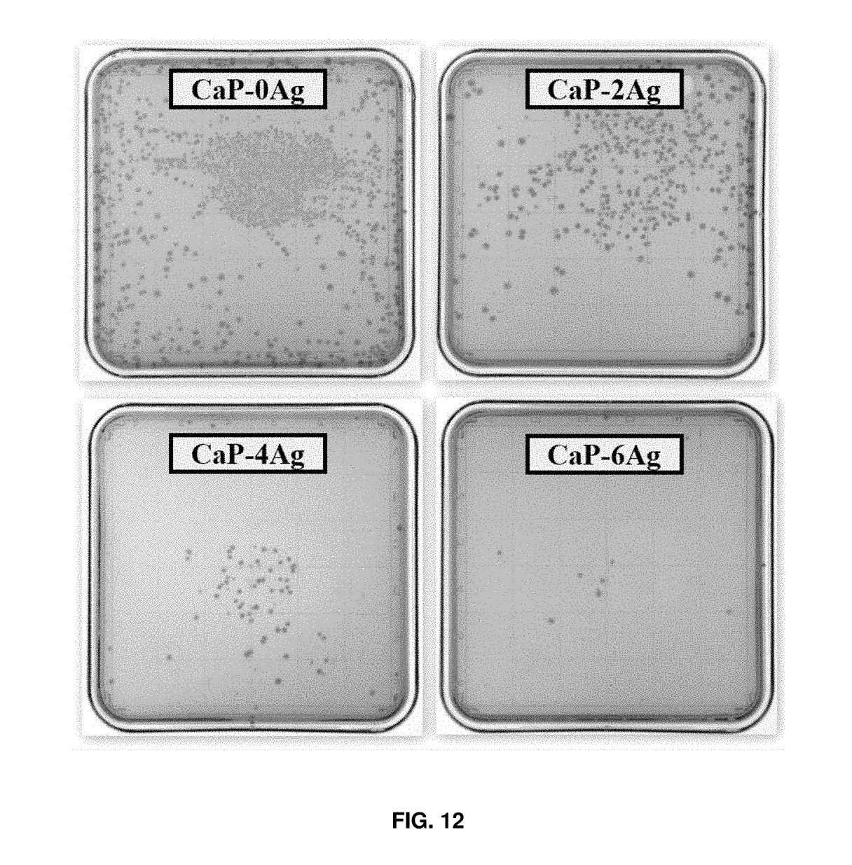

Plate counting results efficiently helped to appraise the antibacterial functionality of the coated specimens. FIG. 11 gives a comparison and percentage decrease in CFUs with respect to the control. CaP-0Ag showed a negligible decrease in CFUs while CaP-2Ag displayed a lesser decrease in CFUs in comparison to other Ag doped samples. The most noteworthy decrease in CFUs was for CaP-6Ag (˜99%). Even CaP-4Ag samples exhibited a significant decrease (˜83%). These results demonstrate the strong ability of the specimens to kill bacteria. FIGS. 12A-12D present the images of the individual agar plates used for the plate counting method. The decrease in the number of CFUs can be clearly seen over an increase in Ag concentration in HA coatings.

Cytotoxicity

The cell proliferation data are presented in FIG. 13. The minimum essential medium alpha (MEM-α) in which the coated samples were incubated for 24 hours was used for the osteoblast cell culture. It is clearly visible that all the coated specimens showed more or almost the same cell viability as the control after 4 days. Clearly, the MC3T3 cells cultured in the medium containing the CaP-0Ag specimen (no Ag doping) exhibited the highest cell viability. This indicates the enhanced biocompatibility of the coatings. In all the other cases, the incorporation of Ag did not affect the cell proliferation at all. Even though a certain concentration of Ag is considered cytotoxic, the Ag doped coated samples in this Example were not harmful to the osteoblasts. Surprisingly, the specimen doped with the highest Ag wt. % (CaP-6Ag) showed higher cell viability than the other two-CaP-0Ag and CaP-4Ag. All over, all the coated Ti6Al4V specimens exhibited satisfactory biocompatibility with no observed cytotoxicity.

### Discussion

Several conclusions can be drawn from the XRD analysis of the microwave processed HA and Ag-HA powders. The diffraction peaks of all the powders were markedly broader, which indicated that both the produced pristine HA and Ag⁺-doped HA powders were nanocrystalline in nature. The crystallite sizes as calculated by the MD JADE software and presented in Table 4 confirm so. A decrease in crystallite size of the powders with increasing amount of Ag doping was also noted. A single diffraction peak can be considered for the crystallite size measurement using Scherrer's formula. In the present Example, the WPF analysis using the MD JADE software considers all the resolvable diffraction peaks and presents a much more reliable set of average crystallite sizes. Further, the XRD patterns of known coatings can be resolved into two different peaks at 2θ angle: ˜28°, ˜29°, whereas, in the present results the peak broadening effect is more pronounced, indicating a higher nanocrystallinity in the microwave processed HA and Ag⁺-doped HA powders.

The slight shift in the diffraction peaks of the Ag doped HA powders, to a lower degree, is interpreted as a substitution effect. This indicates that the microwave processing has helped to successfully dope elements, in this case Ag, into the hexagonal lattice of HA. The shifts in the XRD peaks match with known data.

The impact of incorporating Ag⁺ ions into the HA structure was determined from whole pattern fitting (WPF) and Rietveld refinement of the XRD patterns of the powder samples. The change in the unit cell parameters, specifically, lattice parameters of the Ag⁺-doped HA powders, confirms the previous inference. Without wishing to be bound by theory, it is believed that the inclusion of the Ag⁺ ions with a bigger ionic radius (0.128 nm) into the HA lattice substituted the Ca²⁺ ions with smaller ionic radii (0.099 nm). This difference in the ionic radii of 0.029 nm between Ca²⁺ and Ag⁺ ions is sufficient to distort the lattice parameters. Thus, with an increase of the amount of Ag⁺ ions in the coating composition, the lattice parameters also increased, indicating a pronounced substitutional effect. Without wishing to be bound by theory, it is believed that most of the Ag⁺ ion substituted and occupied Ca²⁺ (4) sites of HA, in good agreement with preferential occupancy of monovalent ions at this specific site of the lattice. However, the Ag⁺ ions can make other site substitutions in the crystal lattice. Thus, after the substitution by Ag⁺ ions, the general formulae of the doped powder composition looks something like Ca₁₀₋ₓAgₓ(PO₄)₆(OH)₂₋ₓ. The vacancy at the hydroxyl site is due to charge imbalance caused by Ag⁺ for Ca²⁺ ions, although PO₄³⁻ for HPO₄²⁻ is also a likely substitution to compensate the charge imbalance.

The formation of other compounds during the synthesis of coatings has been reported. However, the XRD analysis in this Example did not recognize any other phases apart from HA. Even though Ag is entering the HA lattice, it is not diffusing with other elements to form a different compound. This indicates that the amount of Ag doping until 6 wt. % in this case is not saturating the reaction kinetics.

An important condition for an implant material to osseointegrate is to successfully form a biologically active bonelike apatite on its surface. Ti6Al4V is biocompatible but it is not bioactive. To ensure an active substrate surface, the Ti6Al4V substrates were alkali treated (10 M NaOH for 24 hrs at 60° C.) and then heat treated (600° C. for 1 hour) before the actual coating process. Etching results in the formation of negatively charged hydrates which helps in the deposition of a porous sodium titanate hydrogel layer. The subsequent heat treatment at 600° C. dehydrates the hydrogel layer, forming rutile as identified by the XRD. However, when the substrates were immersed in the coating solution, sodium titanate layer was re-hydrated. With the operation of the microwave irradiation, the Na⁺ ions from the substrate surface were rapidly replaced by the H₃O⁺ ions present in the coating solution. This replacement produced Ti—OH groups which became sites for apatite nucleation. With microwave irradiation, two things happened concurrently: a large amount of Ca—P nuclei was formed on the whole substrate surface, and Ag⁺ ions replaced some of the Na⁺ ions and were deposited all over the substrate surface along with Ca²⁺ and PO₄³⁻ ions.

There are two main shortcomings of conventional biomimetic coatings when compared to the present coating technique. First, biomimetic coatings witness growth of Ca—P nuclei into large Ca—P globules over time. The coating developed is more in the perpendicular direction of the substrate than in the horizontal direction (all over the substrate surface). Thus, the specific sites on the substrate surface where the Ca—P nuclei are deposited only have a chance to grow into large globules. Sites devoid of Ca—P nuclei form empty patches. As a result, attaining a homogeneous coating over the substrate becomes more difficult. Microwave irradiation initiates a coating development mechanism which is different from the biomimetic one. When the microwave is in operation, large amounts of independent Ca—P nuclei are formed all over the surface of Ti6Al4V implants and, over time, all of the deposited Ca—P nuclei grow. In this way a more uniform coating covering the whole implant surface is witnessed as confirmed by the SEM images in FIGS. 5A-5D. Even though most of the developed HA was nanocrystalline in nature, the textured orientation of the XRD plots is an indication of the homogeneous development of the coatings on substrates.

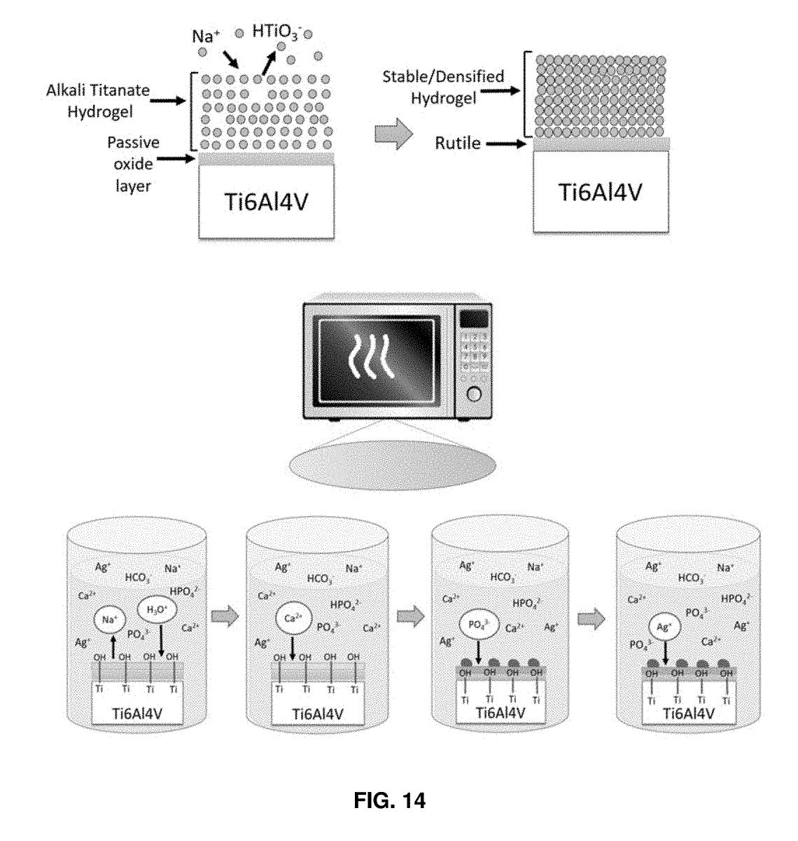

Second, the present Example demonstrates incorporation of bactericidal Ag⁺ ions into Ca—P layer by means of biomimetic coating, which was needed in the art. In this Example, by relaxing the stringent conditions of biomimetics along with the microwave irradiation, Ag⁺ ions were successfully incorporated into the Ca—P layer. The EDS results as shown in FIGS. 6B-6D confirm the successful incorporation of Ag in the coating along with the presence of Ca and P elements. Thus, microwave irradiation resulted in the formation of an ultra-thin Ag incorporated Ca—P coating layer which is bonded to the Ti6Al4V surface by means of hydrated titania and Ti—OH. The difference in coating mechanisms between biomimetic coating and the microwave coating mechanism has been shown. FIG. 14 shows the mechanism followed for the formation of nanocrystalline calcium phosphate layer on Ti6Al4V in this Example and then the incorporation of Ag⁺ ions into it.

The water contact angle measurements show that uncoated Ti6Al4V is hydrophobic in nature (contact angle as high as 76°). The significant decrease in water contact angle ensures that microwave assisted coating successfully deposited HA on the Ti alloy surface. HA is hydrophilic in nature and having a hydrophilic implant surface is extremely desired for efficient osseointegration. The coating thus enhances wettability which in turn accelerates bond binding.

According to the “race for the surface” concept, if the bacterial colonization is more rapid than the host cell attachment, then the risk of surgical site infection becomes quite certain. The appropriate scenario at the time of implantation can be constructed as per the following: Stage 1: A faster attachment of the host cells to the implant surface over the bacteria; Stage 2: In spite of the host cells' colonization, even if there is any bacteria attached onto the surface, the coating on the implants should be capable of killing them; Stage 3: A continuous mechanism should also work at the same time which would kill any bacteria surrounding the implants. In this Example, the as-developed HA layer on Ti6Al4V was so uniform and active that rapid hydration of the oxide layer catalyzed the adhesion of biomolecules (host osteoblast cells) onto the implant surface. Another mechanism may be that a fibrin network was laid upon the TiO₂ surface and the associated absorbed molecules after implantation enhanced the attachment of local osteoblasts in the early healing phase. Thus, the microwave assisted coating helped the host cells to win the race for the surface by making the implant surface hydrophilic and bioactive.

During stage 2, after the attachment of osteoblasts, if there is any presence of bacteria on the implant surface, the coating in this Example is capable of killing them. When the bone apposition layer was attached to the Ti6Al4V substrate surface, simultaneously there was a continuous release of Ag⁺ ions. The ICP results (FIG. 8) show a similar release tendency for all the coatings which contained three different concentrations of Ag. The Ag⁺ ion release for all the samples was a bit high in the first 3 days but it slowly stabilized over the next few days. The release of silver from CaP-2Ag decreased after the 3^{rd} day, but in the case of the other two samples (CaP-4Ag and CaP-6Ag), the release was quite continual and constant over days. This kind of Ag⁺ release around the implants is important for sustainable prevention of bacterial colonization. The ion release trend is also satisfactory. The antibacterial functionality is confirmed by the ZOI and colony forming unit tests. In the ZOI tests, the implants were exposed to a heavy concentration of E. coli bacteria (OD=1.0). Even though the ICP results showed a small concentration of silver release, it was sufficient to kill any bacteria distal to the surface. All the silver doped coated samples formed distinct ZOIs around it while the specimen with only Ca—P coating could not prevent the growth of bacteria around it.

The ZOI coefficient, which is a direct interpretation of the ZOI sizes for different specimens, can be correlated to the ICP results. The ZOI coefficient was the highest (largest ZOI area) in the case of CaP-6Ag when compared to other samples. During the first 3 days, as there was a rise in the silver ion release as shown by ICP, there was a slight increase in the ZOI coefficient. After the 3^{rd} day of incubation, along with the slight drop in Ag⁺ release, the ZOI coefficient also experienced a minute diminishment. In the case of CaP-4Ag samples, the ZOI characteristics were similar to CaP-6Ag, while CaP-2Ag experienced a comparatively pronounced reduction in ZOI along with a similar drop in ion release after the 3^{rd} day. However, all the samples could successfully inhibit bacterial growth around them even after 6 days of incubation. The distinct ZOIs correlating with the release of Ag⁺ ions once again confirmed the sustainable capability of the microwave coated specimens to prevent bacterial growth.

It can also be concluded that the concentration of Ag⁺ ion release is completely proportional to the extent of bacterial resistance. The greater the Ag⁺ release, the greater the ZOI area is, which in turn highlights the extent of bacterial prevention. In the CFU test, all the coated samples were immersed in a specific bacterial dilution for 4 hrs. The specimen coatings doped with the highest percentage of Ag (CaP-6Ag) exhibited the maximum reduction in CFUs followed by CaP-4Ag specimens which also reduced the thriving of CFUs by 83% (FIG. 11). As compared to the other two Ag doped specimens, CaP-2Ag released the lowest amount of Ag and as a result the bactericidal properties were lesser (22%). In both the antibacterial tests, neither could the CaP-0Ag specimens resist bacterial growth around the surface nor could they kill the CFUs. The slight decrease in CFUs in case of CaP-0Ag can be accounted to human errors while visual counting. As each CFU corresponds to a viable bacteria, this test confirms that the silver ions released from the microwave coated Ti6Al4V specimens have the capability of not only resisting bacterial growth but also to kill it if there is a surface contact.

It is known that silver ions and silver compounds are highly toxic to most bacterial strains. Though extensive research has been carried out to understand the silver acting mechanism on bacteria, in most cases it still remains unclear. First of all, bacterial inhibition depends on the concentration of the Ag⁺ solution as well the number of CFUs present in the experiment. In this Example, the concentration of released Ag⁺ ions from the coating and applied CFUs were precise, as all the Ag doped specimens exhibited antibacterial functionality.

Without wishing to be bound by theory, the antibacterial mechanism and possible interaction phenomena of the released Ag⁺ ions from the coating with the E. coli are explained in a few ways. In the first mechanism, an electrostatic attraction develops between the negatively charged bacterial cells and positively charged bactericidal elements. The released silver ions from the microwave assisted coating, irrespective of the charge, firstly attacks the outer surface membrane outside the peptidoglycan layer of E. coli. As soon as the metal ions interact with the membrane, it drains the tightly packed lipopolysaccharide (LPS) and protein molecules present in the membrane structure. This action significantly disrupts the permeability barrier forming irregular sHAed ‘pits’ or ‘holes’ in the E. coli cell membrane. As a result, the membrane permeability increases and the bacterial cells become incapable of regulating the transport through the plasma membrane which is followed by the cell death. Thus, in this mechanism the detrimental increase in membranous permeability is the reason for E. coli growth inhibition. In the second mechanism, the Ag particles break through the barrier of outer membrane permeability, peptidoglycan and periplasm, and destroy the respiratory chain dehydrogenases. As a result, the cellular respiration is inhibited, causing cell death. In the third possible mechanism, Ag⁺ ions interact with the thiol (—SH) group of cysteine by replacing the hydrogen atom to form —S—Ag. Even this disrupts the enzymatic function of the affected protein which in turn inhibits growth of E. coli. In most cases, the action of Ag⁺ can also leave the E. coli bacterial cells in a state of ABNC (active but non-culturable) in which the cells remain capable of metabolic activity but develop an obstinacy to re-generation. All of the above described mechanisms are possible explanations for the antibacterial effect of released Ag⁺ from the microwave assisted coated Ti6Al4V specimens against E. coli.

Preliminary cytocompatibility tests are important to evaluate the biological properties of the coated implants prior to in vivo evaluation. A certain concentration of silver is proven to be cytotoxic in mammalian cells. In this Example, the coated samples were incubated in MEM-α culture medium for 24 hrs and the collected extracts were used to culture MC3T3-E1 cells. Even after 4 days, the cell viability of all the coated samples were more or almost the same as the control. Even the cell viability of specimens coated with 6 wt. % Ag were almost similar to the specimen, which contained no silver in their coatings. This corroborates that the concentration of silver ion release in this Example is not enough to induce cytotoxicity, though it is sufficient to kill bacteria. The cytotoxicity test also confirms that the Ca—P nuclei formed on the substrates as a result of the microwave irradiation enhances bone apposition. These findings lead to the conclusion of achieving “bifunctional” coatings of Ag⁺-doped HA.

Conclusion

The results presented in this Example dictate the conclusion that successful ‘bifunctional’ Ag⁺-doped HA coatings on Ti6Al4V substrates can be developed by the process described herein. Microwave irradiation proves to be an efficient technique to form coatings on the implant substrate, which are capable of osseointegration synchronized with antibacterial functionality. The microwave was used to develop antibacterial coatings on biomaterials with advantages that include a uniform coating over the implant surface, controlled release of Ag⁺ ions from the coating, s rapid coating process, and low processing costs.

### Example II—Magnesium Phosphate Doped with Silver

MgP coatings were prepared in the same manner as described in Example I for HA coatings. Specifically, newberyite coatings were prepared on Ti6Al4V substrates with varying amounts of silver as follows (where the amount of Ag is described in wt. % relative to the weight of the source of Mg ions in the coating solution): MgP-0Ag, MgP-1Ag, MgP-3Ag, and MgP-5Ag.

The reagents used in this Example were magnesium nitrate hexahydrate (Mg(NO₃)₂.6H₂O, 98% purity) procured from Alfa Aesar, sodium phosphate monobasic anhydrous (NaH₂PO₄, >98% purity) and sodium hydroxide reagent grade (NaOH, ≥97% purity) procured from Fischer Scientific, and silver nitrate (AgNO₃, ≥99% purity) from Sigma Aldrich. The coating solutions containing varying percentages of Ag were prepared as per the following recipe. The above-described reagents were added one by one into a 250 ml pyrex glass beaker containing 200 ml distilled water and mixed properly using a teflon coated magnetic stirrer at 260 rpm. The pH values of all the coating solutions were adjusted to 5.6 by adding 2 ml of 1M NaOH solution. After proper dissolution of all the reagents, two Ti6Al4V blocks were placed in a 200 ml beaker containing 100 ml of the coating solution covered with a glass plate to avoid spilling. The whole set-up was moved into a 1200 W microwave oven (Panasonic) and irradiated at the highest power for 5 mins. At the end of one cycle, 100 ml of the remaining coating solution was added and irradiated for another 5 mins.

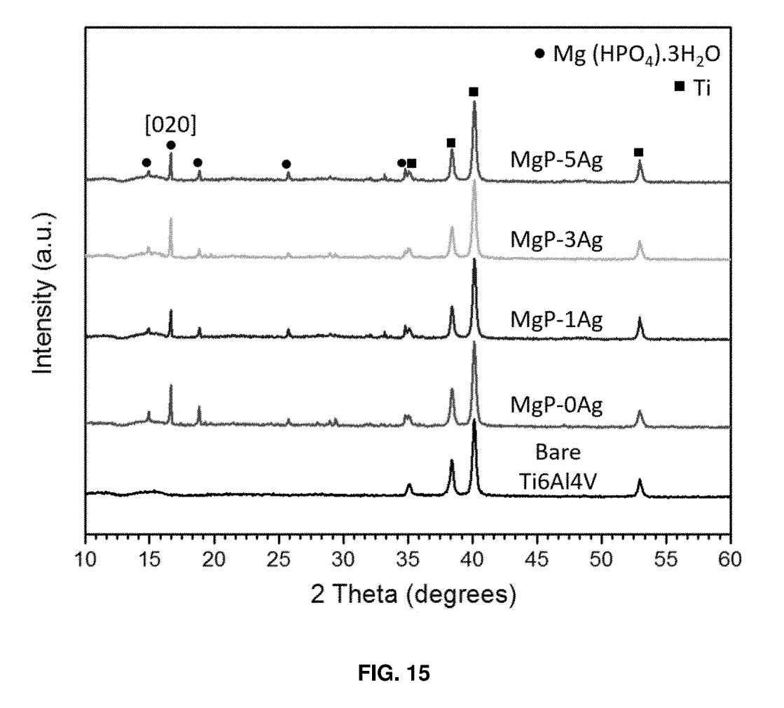

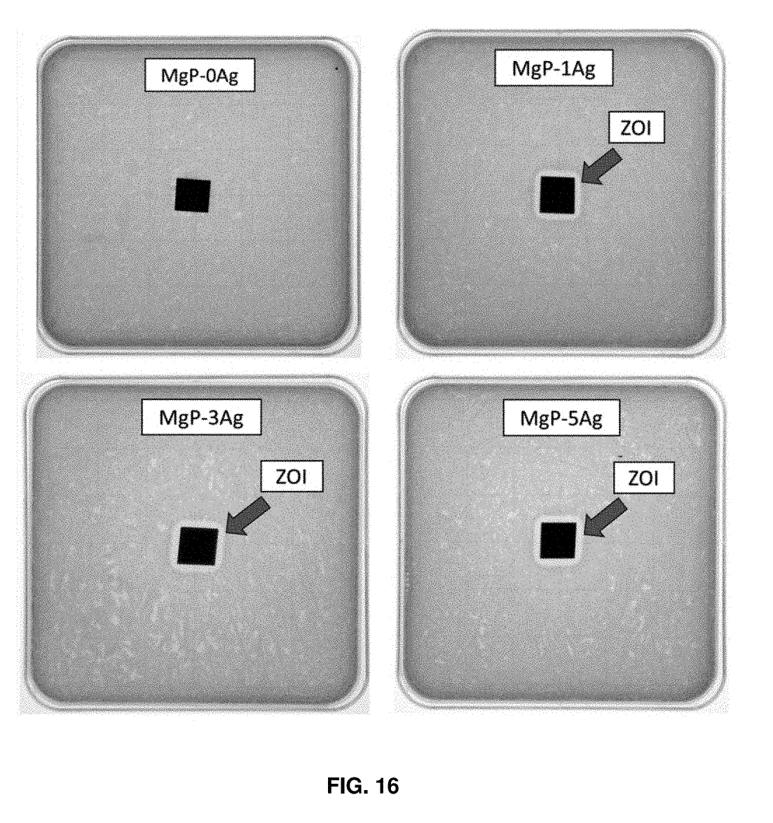

The coatings were characterized as before. FIG. 15 shows the XRD patterns of these coating compositions. Notably, the XRD patterns do not include any peaks attributable to elemental silver. The coatings were subjected to in-vitro test assays such as zone of inhibition (ZOI) tests, and counting of colony forming units (CFUs), to evaluate the antibacterial characteristics of the coatings against E. coli. FIG. 16 shows the images of the individual agar plates used for the plate counting method. The decrease in the number of CFUs can be clearly seen over an increase in Ag concentration in the MgP coatings. FIG. 16 also shows that MgP without silver did not produce a zone of inhibition. Thus, the MgP coating by itself (i.e., without silver) is not antibacterial.

Certain embodiments of the compositions and methods disclosed herein are defined in the above examples. It should be understood that these examples, while indicating particular embodiments of the invention, are given by way of illustration only. From the above discussion and these examples, one skilled in the art can ascertain the essential characteristics of this disclosure, and without departing from the spirit and scope thereof, can make various changes and modifications to adapt the compositions and methods described herein to various usages and conditions. Various changes may be made and equivalents may be substituted for elements thereof without departing from the essential scope of the disclosure. In addition, many modifications may be made to adapt a particular situation or material to the teachings of the disclosure without departing from the essential scope thereof.

What is claimed is:

1. A composition comprising a phosphate material doped with Ag, wherein the phosphate material is selected from the group consisting of hydroxyapatite and newberyite, and wherein the composition is characterized by an X-ray diffraction pattern with no peaks corresponding to elemental Ag.

2. The composition of claim 1, wherein the composition is a coating on a substrate comprising Ti.

3. The composition of claim 2, further comprising a TiO₂ phase on the substrate.

4. The composition of claim 3, wherein the TiO₂ phase is rutile.

5. A method of making a coating on a substrate, the method comprising:
immersing a substrate in a coating solution comprising a source of silver ions; and
exposing the immersed substrate to microwave radiation so as to form a silver-containing coating on the substrate, wherein the silver-containing coating comprises calcium phosphate or magnesium phosphate.

6. The method of claim 5, wherein the coating solution further comprises a source of calcium ions and a source of phosphate ions.

7. The method of claim 6, wherein the source of calcium ions comprises a water-soluble calcium-containing compound selected from the group consisting of: calcium nitrate [Ca(NO₃)₂], calcium chloride (CaCl₂), calcium bromide (CaBr₂), calcium acetate [(CH₃COO)₂Ca], calcium citrate, calcium iodide (CaI₂), calcium lactate, calcium gluconate, calcium fumarate, calcium oxide (CaO), calcium hydroxide [Ca(OH)₂], calcium benzoate, calcium formate, calcium butyrate, calcium isobutyrate, calcium malate, calcium maleate, calcium propionate, calcium valerate, hydrates thereof, and combinations thereof.

8. The method of claim 6, wherein the source of phosphate ions is selected from the group consisting of: phosphoric acid (H₃PO₄), ammonium dihydrogen phosphate (NH₄H₂PO₄), sodium dihydrogen phosphate (NaH₂PO₄), lithium dihydrogen phosphate (LiH₂PO₄), potassium dihydrogen phosphate (KH₂PO₄), cesium dihydrogen phosphate (CsH₂PO₄), rubidium dihydrogen phosphate (RbH₂PO₄), and combinations thereof.

9. The method of claim 5, wherein the coating solution further comprises a source of magnesium ions and a source of phosphate ions.

10. The method of claim 9, wherein the source of magnesium ions comprises a water-soluble magnesium-containing compound selected from the group consisting of: magnesium nitrate [Mg(NO₃)₂], magnesium chloride (MgCl₂), magnesium sulfate (MgSO₄), magnesium acetate, magnesium benzoate, magnesium bromide (MgBr₂), magnesium chromate, magnesium bromate, magnesium citrate, magnesium formate, magnesium hexafluorosilicate, magnesium iodide (MgI₂), magnesium lactate, magnesium perchlorate, magnesium salicylate, magnesium sulfite, magnesium tartrate, magnesium thiosulfate, hydrates thereof, and combinations thereof.

11. The method of claim 9, wherein the source of phosphate ions is selected from the group consisting of: phosphoric acid (H₃PO₄), ammonium dihydrogen phosphate (NH₄H₂PO₄), sodium dihydrogen phosphate (NaH₂PO₄), lithium dihydrogen phosphate (LiH₂PO₄), potassium dihydrogen phosphate (KH₂PO₄), cesium dihydrogen phosphate (CsH₂PO₄), rubidium dihydrogen phosphate (RbH₂PO₄), and combinations thereof.

12. The method of claim 5, wherein the coating solution further comprises a pH buffer.

13. The method of claim 5, wherein the substrate is an orthopedic or dental implant.

14. The method of claim 5, wherein the substrate comprises titanium, gold, silver, stainless steel, tantalum, platinum, tungsten, palladium, chromium, cobalt, alumina, zirconia, polyetheretherketone (PEEK), ultra high molecular weight polyethylene (UHMWPE), polymethyl methacrylate (PMMA), polylactic acid (PLA), polylactic acid co-polymer with glycolic acid (PLGA), polycaprolactone (PCL), polyhydroxyalkanoates (PHA) such as poly-3-hydroxybutyrate (P3HB), or mixtures, composites, or combinations thereof.

15. The method of claim 5, wherein the substrate comprises Ti6Al4V.

16. The method of claim 5, wherein the source of silver ions comprises a water-soluble silver compound selected from the group consisting of: silver nitrate (AgNO₃), silver chloride (AgCl), silver fluoride (AgF), silver acetate (AgC₂H₃O₂), silver permanganate (AgMnO₄), silver sulfate (Ag₂SO₄), silver nitrite (AgNO₂), silver bromated (AgBrO₃), silver salicylate (HOC₆H₄COOAg), silver iodate (AgIO₃), silver dichromate (Ag₂Cr₂O₇), silver chromate (Ag₂CrO₄), silver carbonate (Ag₂CO₃), silver citrate (C₆H₈Ag₃O₇), silver phosphate (Ag₃PO₄), silver stearate (Ag[(CH₃)(CH₂)]₁₆)CO₂), silver (I) oxide (Ag₂O), silver sulfide (Ag₂S), silver bromide (AgBr), silver iodide (AgI), silver cyanide (AgCN), and combinations thereof.

17. The method of claim 5, further comprising an etching step prior to the immersing, wherein the substrate is etched with NaOH.

18. The method of claim 17, further comprising rinsing the etched substrate with water, drying the rinsed substrate, and cooling the dry substrate to room temperature prior to the immersing.

19. The method of claim 5, further comprising drying the silver-containing coating.

20. The product of the method of claim 5.

21. An orthopedic or dental implant comprising:
a substrate; and
a bifunctional coating on the substrate, wherein the bifunctional coating comprises a silver-doped calcium phosphate or a silver-doped magnesium phosphate, the bifunctional coating being characterized by an X-ray diffraction pattern having no peaks attributal to elemental silver;
wherein the bifunctional coating is antibacterial and capable of osseointegration.

22. The orthopedic or dental implant of claim 21, wherein the bifunctional coating is not cytotoxic.

23. The orthopedic or dental implant of claim 21, wherein the silver-doped calcium phosphate is silver-doped hydroxyapatite, or wherein the silver-doped magnesium phosphate is silver-doped newberyite.

24. The orthopedic or dental implant of claim 21, wherein the substrate comprises titanium, gold, silver, stainless steel, tantalum, platinum, tungsten, palladium, chromium, cobalt, alumina, zirconia, polyetheretherketone (PEEK), ultra high molecular weight polyethylene (UHMWPE), polymethyl methacrylate (PMMA), polylactic acid (PLA), polylactic acid co-polymer with glycolic acid (PLGA), polycaprolactone (PCL), polyhydroxyalkanoates (PHA) such as poly-3-hydroxybutyrate (P3HB), or mixtures, composites, or combinations thereof.
